(19) United States
(12) Patent Application Publication
(10) Pub. No.: US 2017/0260126 A1
(43) Pub. Date: Sep. 14, 2017

(54) SQUALENE COMPOUNDS AS MODULATORS OF LDL-RECEPTOR EXPRESSION

(71) Applicant: OXFORD UNIVERSITY INNOVATION LIMITED, Oxford (GB)

(72) Inventors: Graham Michael Wynne, Oxford (GB); Angela Jane Russell, Oxford (GB); Joseph Mason, Oxford (GB); Carole Jeanne Rachele Bataille, Oxford (GB); Richard Wade-Martins, Oxford (GB); Alastair Kerr, Oxford (GB); Olivia Hibbitt, Oxford (GB); Lawrence Tam, Oxford (GB)

(21) Appl. No.: 15/510,836

(22) PCT Filed: Sep. 15, 2015
(86) PCT No.: PCT/GB2015/052673

§ 371 Date: Mar. 13, 2017

(30) Foreign Application Priority Data
Sep. 15, 2014 (GB) 1416273.9

(51) Int. Cl. C07C 217/18; C07C 43/23; C07C 39/21; C07C 43/215; C07C 217/20

(52) U.S. Cl. CPC C07C 217/18; C07C 43/215; C07C 217/20; C07C 39/21; C07C 43/23

(57) ABSTRACT

The present invention relates to compounds that modify low density lipoprotein receptor (LDLR) expression. The compounds have the structural formula I shown below:

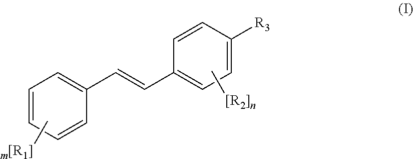

wherein m, R₁, n, R₂ and R₃ are each as defined herein. The present invention also relates to processes for the preparation of these compounds, to pharmaceutical compositions comprising them, and to their use in the treatment of diseases or disorders associated with elevated levels of low density lipoprotein cholesterol (LDL-C).

The present invention relates to therapeutic compounds. More specifically, the present invention relates to compounds that modify low density lipoprotein receptor (LDLR) expression. The present invention also relates to processes for the preparation of these compounds, to pharmaceutical compositions comprising them, and to their use in the treatment of diseases or disorders associated with elevated levels of low density lipoprotein cholesterol (LDL-C).

### BACKGROUND OF THE INVENTION

The LDL receptor (LDLR) is of key importance in the cellular binding and subsequent removal of circulating plasma cholesterol.⁴ The expression of the LDL receptor gene is carefully regulated by means of a Sterol Regulatory Element Binding Protein (SREBP). Low LDL cholesterol concentrations inside the cell lead to upregulation of receptor synthesis to give increased uptake of LDL cholesterol from the bloodstream.⁵ Mutations to the gene responsible for encoding the LDL receptor may cause either loss of synthesis of the receptor, or production of receptors which fail to bind and internalise LDL cholesterol.⁶

These mutations are the leading cause of familial hypercholesterolaemia (FH),⁴ a hereditary condition that has an estimated prevalence of approximately 1 in 500 for the heterozygous condition, and up to 1 in one million in the case of homozygous FH.⁷ Characterised by an increase in circulating LDL cholesterol,⁵ FH carries a greater risk of cardiovascular disease,⁹ the leading cause of non-communicable disease death worldwide.⁹ Atherosclerosis and other related symptoms can occur,¹⁰ which will eventually lead to increasingly severe cardiovascular events such as myocardial infarctions.⁵ These clinical manifestations occur in greater than 50% of untreated males with FH before 50 years of age, and at least 30% of females by the age of 60.¹¹

For patients with heterozygous FH, upregulation of the LDLR gene can produce enough receptor to compensate the fact that only half are truly functional. Expression of the LDL receptor is tightly controlled by the SREBP⁵ meaning the focus of research designed to upregulate the LDL receptor to date has been on inhibiting the biosynthetic pathway of acetyl coenzyme A to cholesterol. Cholesterol can then be removed from the bloodstream and excreted via bile acids into the intestine.¹² Two key steps in the pathway have been the focus of study: the conversion of 3-hydroxy-3-methylglutaryl coenzyme A (HMG-CoA) to mevalonate, and the head-to-head conversion of farnesyl pyrophosphate (FPP) into squalene.

The conversion of HMG-CoA to mevalonate is the rate limiting step in this pathway', and currently remains the only clinically useful point to inhibit the biosynthesis of cholesterol.¹⁵ Statins, inhibitors of the enzyme HMG-CoA reductase, are therefore the current treatment of choice for heterozygous FH.¹¹

Lovastatin was originally isolated from the fungus Aspergillus terreus, and went on to become the first statin approved for use as an HMG-CoA reductase inhibitor¹⁶. Modifications have been made since then to limit the side-effects and improve therapeutic action, to produce a range of both fully synthetic and semi-synthetic derivatives, each containing the δ,β-dihydroxycarboxylic acid pharmacophore to mimic HMG-CoA to allow competitive inhibition of HMG-CoA reductase.¹⁷ There are currently five different statins approved for use in the UK: atorvastatin, pravastatin, fluvastatin, simvastatin and rosuvastatin, the structures on which are shown below.¹⁸ These are typically prescribed in conjunction with changes to lifestyle such as increased physical activity, diet modifications and smoking cessation.¹¹ Long term studies have shown that immediate treatment with statins as opposed to delayed treatment results in a 76% reduction in risk of coronary heart disease with the absolute risk of myocardial infarction brought down a level close to that seen in the general population.¹⁹

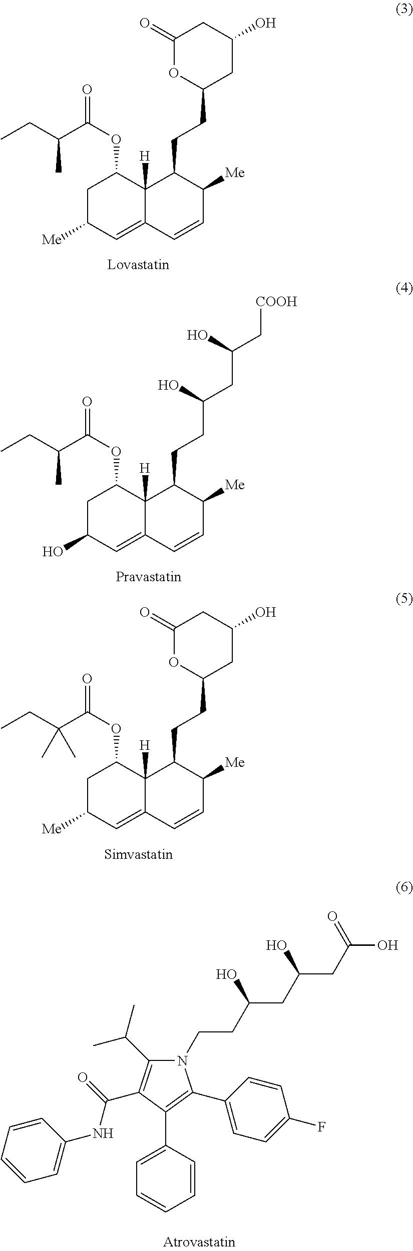 [SMILES: CC[C@H](C)C(=O)O[C@H]1C[C@@H](C)C=C2C=C[C@H](C)[C@H](CC[C@@H]3C[C@@H](O)CC(=O)O3)[C@H]21 ; CC[C@H](C)C(=O)O[C@H]1C[C@H](O)C=C2C=C[C@H](C)[C@H](CC[C@@H](O)C[C@@H](O)CC(=O)O)[C@H]21 ; CCC(C)(C)C(=O)O[C@H]1C[C@@H](C)C=C2C=C[C@H](C)[C@H](CC[C@@H]3C[C@@H](O)CC(=O)O3)[C@H]21 ; CC(C)c1c(C(=O)Nc2ccccc2)c(-c2ccccc2)c(-c2ccc(F)cc2)n1CC[C@@H](O)C[C@@H](O)CC(=O)O]

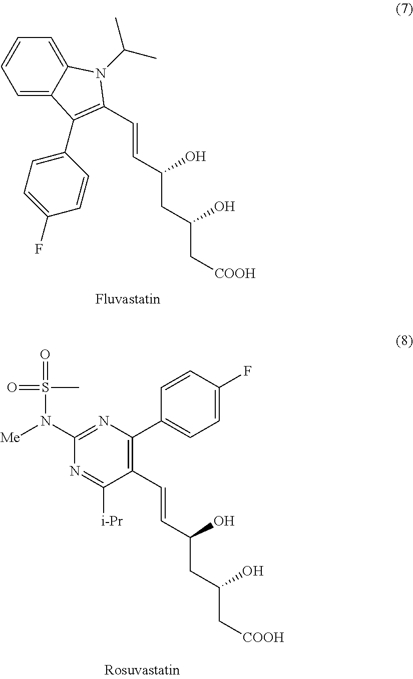 [SMILES: CC(C)n1c(/C=C/[C@H](O)C[C@H](O)CC(=O)O)c(-c2ccc(F)cc2)c2ccccc21 ; CC(C)c1nc(N(C)S(C)(=O)=O)nc(-c2ccc(F)cc2)c1/C=C/[C@@H](O)C[C@H](O)CC(=O)O]

However, 27% of individuals on maximum lipid lowering statin therapy still fail to achieve the recommended LDL cholesterol level of ≦2.5 mmol/L, and of those on any kind of statin regimen, only 47% reach a desirable target.²⁰ Furthermore, there is strong evidence to suggest that an even lower target would further decrease the occurrence of clinical events in patients,²¹⁻²³ giving rise to the maxim “lower is better” in lipid management.¹⁵ Finally, statins have been repeatedly linked with side-effects such as myopathy,²⁰ potentially affecting up to 20% of those taking them²⁴; this results in poor adherence by patients, who are subsequently less likely to achieve the target lipid levels.²⁵

The issues encountered with statins have triggered research into inhibition of an alternative step: squalene production from farnesyl pyrophosphate (Scheme 0). Since squalene synthesis occurs further down the acetyl-coA biological pathway, it has been hypothesised that its inhibition may block fewer downstream products and reduce the number of side-effects.^{26,27}

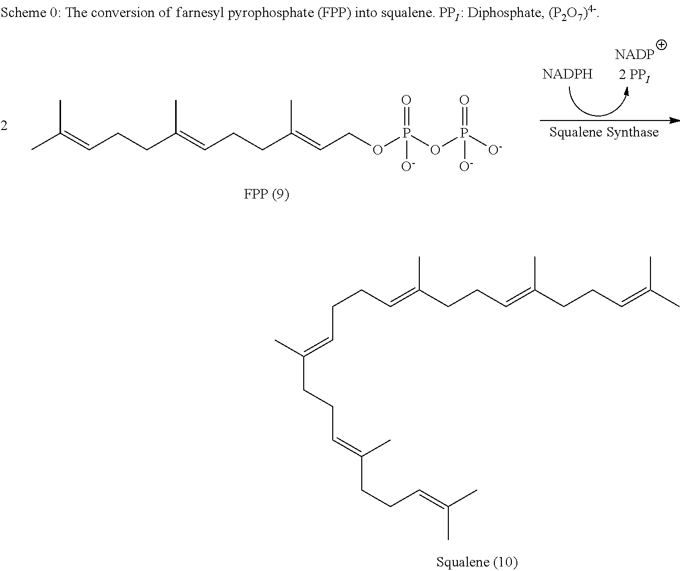

A number of compounds have been developed as squalene synthase inhibitors,^{2,28, 29} notably lapaquistat acetate, which progressed to phase 3 clinical trials, but was discontinued due to hepatic safety issues thought to be unrelated to the inhibition of squalene synthase.³⁰ Thus far, no squalene synthase inhibitor has been approved for medical use; nevertheless, it remains a promising area for research.

Since there is no treatment that currently offers all that is required, it can be concluded (in line with a variety of reviews),^{5,7,15, 32} that there is a definite need for new treatments for familial hypercholesterolaemia, either in conjunction with statins or as a monotherapy.

### SUMMARY OF THE INVENTION

In one aspect, the present invention provides a compound, or a pharmaceutically acceptable salt or solvate thereof as defined herein.

In another aspect, the present invention provides a pharmaceutical composition comprising a compound of the invention as defined herein, or a pharmaceutically acceptable salt or solvate thereof, and one or more pharmaceutically acceptable excipients.

In another aspect, the present invention relates to a compound of the invention as defined herein, or a pharmaceutically acceptable salt or solvate thereof, or a pharmaceutical composition as defined herein, for use in therapy.

In another aspect, the present invention relates to a compound of the invention as defined herein, or a pharmaceutically acceptable salt or solvate thereof, or a pharmaceutical composition as defined herein, for use in the treatment of hypercholesterolaemia.

In another aspect, the present invention relates to the use of a compound of the invention as defined herein, or a pharmaceutically acceptable salt or solvate thereof, in the manufacture of a medicament for use in the treatment of hypercholesterolaemia.

In another aspect, the present invention relates to a method of treating hypercholesterolaemia, said method comprising administering to a subject in need of such treatment a therapeutically effective amount of a compound of the invention as defined herein, or a pharmaceutically acceptable salt or solvate thereof, or a pharmaceutical composition as defined herein.

In another aspect, the present invention provides a compound, or a pharmaceutically acceptable salt or solvate thereof, or a pharmaceutical composition as defined herein, for use in the treatment of diseases or conditions associated with hypercholesterolamia.

Examples of such conditions that are associated with hypercholesterolaemia include atherosclerosis (i.e. atherosclerotic blood lesions) and theft secondary diseases, for example, coronary arterial diseases, cerebral ischemia, intermittent claudication and gangrene.

In another aspect, the present invention provides the use of a compound, or a pharmaceutically acceptable salt or solvate thereof, in the manufacture of a medicament for use in the treatment of for use in the treatment of diseases or conditions associated with hypercholesterolamia.

In another aspect, the present invention provides a method of treating diseases or conditions associated with hypercholesterolemia, said method comprising administering to a subject in need of such treatment a therapeutically effective amount of a compound, or a pharmaceutically acceptable salt or solvate thereof, or a pharmaceutical composition as defined herein.

In another aspect, the present invention provides a compound, or a pharmaceutically acceptable salt or solvate thereof, or a pharmaceutical composition as defined herein, for use in the production of a squalene synthetase inhibitory effect.

In another aspect, the present invention provides the use of a compound, or a pharmaceutically acceptable salt or solvate thereof, in the manufacture of a medicament for use in the production of a a squalene synthetase inhibitory effect.

In another aspect, the present invention provides a method of producing a squalene synthetase inhibitory effect in vitro, said method comprising administering an effective amount of a compound, or a pharmaceutically acceptable salt or solvate thereof.

In another aspect, the present invention provides a method of producing an squalene synthetase inhibitory effect in vivo, said method comprising administering an effective amount of a compound, or a pharmaceutically acceptable salt or solvate thereof.

In another aspect, the present invention relates to a compound of the invention as defined herein, or a pharmaceutically acceptable salt or solvate thereof, or a pharmaceutical composition as defined herein, for use in lowering of blood LDL-C.

In another aspect, the present invention relates to the use of a compound of the invention as defined herein, or a pharmaceutically acceptable salt or solvate thereof, in the manufacture of a medicament for use in lowering of blood LDL-C.

In another aspect, the present invention relates to a method of lowering of blood LDL-C, said method comprising administering to a subject in need of such treatment a therapeutically effective amount of a compound of the invention as defined herein, or a pharmaceutically acceptable salt or solvate thereof, or a pharmaceutical composition as defined herein.

The present invention further provides a method of synthesising a compound, or a pharmaceutically acceptable salt or solvate thereof, as defined herein.

In another aspect, the present invention provides a compound, or a pharmaceutically acceptable salt or solvate thereof, obtainable by, or obtained by, or directly obtained by a method of synthesis as defined herein.

In another aspect, the present invention provides novel intermediates as defined herein which are suitable for use in any one of the synthetic methods set out herein.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

The term “halo” refers to fluoro, chloro, bromo and iodo.

The term “haloalkyl” or “haloalkoxy” is used herein to refer to an alkyl or alkoxy group respectively in which one or more hydrogen atoms have been replaced by halogen (e.g. fluorine) atoms. Examples of haloalkyl and haloalkoxy groups include fluoroalkyl and fluoroalkoxy groups such as —CHF₂, —CH₂CF₃, or perfluoroalkyl/alkoxy groups such as —CF₃, —CF₂CF₃ or —OCF₃.

The term “heterocyclyl”, “heterocyclic” or “heterocycle” means a non-aromatic saturated or partially saturated monocyclic, fused, bridged, or spiro bicyclic heterocyclic ring system(s). Unless otherwise stated herein, monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. Suitably, the term “heterocyclyl”, “heterocyclic” or “heterocycle” will refer to 4, 5 or 6 membered monocyclic rings as defined above.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In a first aspect, the present invention provides a compound of Formula I for use in the treatment of a disease or condition as defined herein (e.g. hypercholesterolaemia)

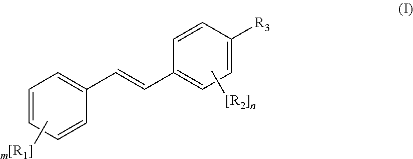

wherein:
m is 0, 1 or 2;
each R₁ group present is independently selected from halo, cyano, nitro, hydroxyl, (1-3C)alkyl, (1-3C)haloalkyl, (1-3C)alkoxy, (1-3C)haloalkoxy, —S—R^{a}, —S(O)—R^{a}, —S(O)₂—R^{a}, —C(O)NR^{a}R^{b}, —NR^{a}C(O)R^{b}, —C(O)R^{a}, —C(O)OR^{a}, —OC(O)R^{a}, —S(O)₂—NR^{a}R^{b} or —NR^{a}S(O)₂R^{b}, wherein R^{a} and R^{b} are each independently selected from hydrogen or (1-3C)alkyl;
n is 0, 1 or 2;
each R₂ group present is independently selected from halo, cyano, nitro, hydroxyl, (1-3C)alkyl, (1-3C)haloalkyl, (1-3C)alkoxy, (1-3C)haloalkoxy, —S—R^{c}, —S(O)—R^{c}, —S(O)—R^{c}, —S(O)₂—R^{c}, —C(O)NR^{c}R^{d}, —NR^{c}C(O)R^{d}, —C(O)R^{c}, —C(O)OR^{c}, —OC(O)R^{c}, —S(O)₂—NR^{c}R^{d} or —NR^{c}S(O)₂R^{d}, wherein R^{c} and R^{d} are each independently selected from hydrogen or (1-3C)alkyl; and

R₃ is selected from hydrogen, hydroxyl, (1-3C)alkoxy, halo, cyano, nitro or a group of the formula:

—X¹-L-X²-Q

wherein:
X¹ is absent or selected from —O—, —N(R^{e})—, —N(R^{e})—C(O)—, —C(O)—N(R^{e})—, —N(R^{e})C(O)N(R^{f})—, —C(O)—, —C(O)O—, —OC(O)—, —S—, —SO—, —SO₂—, —S(O)₂N(R^{e})—, —N(R^{e})SO₂—, wherein R^{e} and R^{f} are each independently selected from hydrogen or (1-3C)alkyl;
L is —(CR^{g}R^{h})ₚ—, wherein p is 3, 4 or 5, and R^{g} and R^{h} are at each occurrence independently selected from hydrogen, (1-2C)alkyl or (1-2C)haloalkyl, or R^{g} and R^{b} can be linked such that, together with the carbon atom to which they are attached, they form a (3-6C)cycloalkyl ring;
X² is absent or selected from —O—, —N(Rⁱ)—, —N(Rⁱ)—C(O)—, —C(O)—N(Rⁱ)—, —N(R^{j})C(O)N(Rⁱ)—, —C(O)—, —C(O)O—, —OC(O)—, —S—, —SO—, —SO₂—, —S(O)₂N(Rⁱ)—, —N(Rⁱ)SO₂—, wherein Rⁱ and R^{j} are each independently selected from hydrogen or (1-3C)alkyl; and
Q is selected from hydrogen or (1-6C)alkyl, wherein, when Q is (2-6C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;
or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)haloalkyl, (1-2C)alkoxy or (1-2C)haloalkoxy;
   or a pharmaceutically acceptable salt or solvate thereof.

In another aspect, the present invention provides a compound of formula I for use in the treatment of a disease or condition as defined herein (e.g. hypercholesterolaemia) with the proviso that Q and Rⁱ are not both ethyl when X¹ is —O—, p is 3, R^{g} and R^{h} are both hydrogen; and X² is —N(Rⁱ)—.

Suitably, Q and Rⁱ are both hydrogen or methyl when X¹ is —O—, p is 3, R^{g} and R^{h} are both hydrogen; and X² is —N(Rⁱ)—.

Suitably, Q is methyl and Rⁱ is hydrogen or methyl.

Particular compounds of the invention include, for example, compounds of the formula I, or pharmaceutically acceptable salts thereof, wherein, unless otherwise stated, each of m, n, R₁, R₂, R₃, X¹, L, X² and Q has any of the meanings defined hereinbefore or in any of paragraphs (1) to (42) hereinafter:
(1) m is 0 or 1;
(2) m is 0;
(3) m is 1;
(4) m is 2;
(5) each R₁ group present is independently selected from halo, cyano, nitro, hydroxyl, (1-2C)alkyl, (1-2C)haloalkyl, (1-2C)alkoxy, (1-2C)haloalkoxy, —S—R^{a}, —S(O)—R^{a}, —S(O)₂—R^{a}, —C(O)NR^{a}R^{b}, —NR^{a}C(O)R^{b}, —C(O)R^{a}, —C(O)OR^{a}, —OC(O)R^{a}, —S(O)₂—NR^{a}R^{b} or —NR^{a}S(O)₂R^{b}, wherein R^{a} and R^{b} are each independently selected from hydrogen or (1-2C)alkyl;
(6) each R₁ group present is independently selected from halo, cyano, nitro, hydroxyl, (1-2C)alkyl, (1-2C)haloalkyl, (1-2C)alkoxy, (1-2C)haloalkoxy, —S(O)₂—R^{a}, —C(O)NR^{a}R^{b}, —NR^{a}C(O)R^{b}, —C(O)OR^{a}, —OC(O)R^{a}, —S(O)₂—NR^{a}R^{b} or —NR^{a}S(O)₂R^{b}, wherein R^{a} and R^{b} are each independently selected from hydrogen or (1-2C)alkyl;
(7) each R₁ group present is independently selected from halo, cyano, nitro, hydroxyl, (1C)alkyl, (1C)haloalkyl, (1C)alkoxy, (1C)haloalkoxy, —S(O)₂—R^{a}, —C(O)NR^{a}R^{b}, —NR^{a}C(O)R^{b}, —C(O)OR^{a}, —OC(O)R^{a}, —S(O)₂—NR^{a}R^{b} or -NR^{a}S(O)₂R^{b}, wherein R^{a} and R^{b} are each independently selected from hydrogen or (1C)alkyl;
(8) each R₁ group present is independently selected from halo, cyano, nitro, hydroxyl, (1-2C)alkyl, (1-2C)haloalkyl, (1-2C)alkoxy or (1-2C)haloalkoxy;
(9) each R1 group present is independently selected from fluoro, chloro, cyano, nitro, hydroxyl, (1-2C)alkyl, (1-2C)haloalkyl, (1-2C)alkoxy or (1-2C)haloalkoxy;
(10) each R1 group present is independently selected from fluoro, chloro, cyano, nitro, hydroxyl, methyl, trifluoromethyl, methoxy or trifluoromethoxy;
(11) each R₁ group present is independently selected from fluoro, cyano, nitro, hydroxyl, methyl, or methoxy;
(12) n is 0 or 1;
(13) n is 0;
(14) n is 1;
(15) n is 2;
(16) each R₂ group present is independently selected from halo, cyano, nitro, hydroxyl, (1-2C)alkyl, (1-2C)haloalkyl, (1-2C)alkoxy, (1-2C)haloalkoxy, —S—R^{c}, —S(O)—R^{c}, —S(O)₂—R^{c}, —C(O)NR^{c}R^{d}, —NR^{c}C(O)R^{d}, —C(O)R^{c}, —C(O)OR^{c}, —OC(O)R^{c}, —S(O)₂—NR^{c}R^{d} or —NR^{c}S(O)₂R^{d}, wherein R^{c} and R^{d} are each independently selected from hydrogen or (1-2C)alkyl;
(17) each R₂ group present is independently selected from halo, cyano, nitro, hydroxyl, (1C)alkyl, (1C)haloalkyl, (1C)alkoxy, (1C)haloalkoxy, —S—R^{c}, —S(O)—R^{c}, —S(O)₂—R^{c}, —C(O)NR^{c}R^{d}, —NR^{c}C(O)R^{d}, —C(O)R^{c}, —C(O)OR^{c}, —OC(O)R^{c}, —S(O)₂—NR^{c}R^{d} or —NR^{c}S(O)₂Rd, wherein R^{c} and R^{d} are each independently selected from hydrogen or (1C)alkyl;
(18) each R₂ group present is independently selected from halo, cyano, nitro, hydroxyl, (1-2C)alkyl, (1-2C)haloalkyl, (1-2C)alkoxy, (1-2C)haloalkoxy, —S—R^{c}, —S(O)—R^{c}, —S(O)₂—R^{c}, —C(O)NR^{c}R^{d}, —NR^{c}C(O)R^{d}, —C(O)R^{c}, —C(O)R^{c}, —OC(O)R^{c}, —S(O)₂—NR^{c}R^{d} or —NR^{c}S(O)₂R^{d}, wherein R^{c} and R^{d} are each independently selected from hydrogen or (1-2C)alkyl;
(19) each R₂ group present is independently selected from halo, cyano, nitro, hydroxyl, (1-2C)alkyl, (1-2C)haloalkyl, (1-2C)alkoxy, (1-2C)haloalkoxy;
(20) each R₂ group present is independently selected from fluoro, chloro, cyano, nitro, hydroxyl, (1-2C)alkyl, (1-2C)haloalkyl, (1-2C)alkoxy, (1-2C)haloalkoxy;
(21) each R₂ group present is halo (e.g. fluoro);
(22) each R₂ group present is fluoro;
(23) R₃ is selected from hydroxyl, (1-2C)alkoxy, or a group of the formula:

—X¹-L-X²-Q

wherein:

X¹ is selected from —O—, —N(R^{e})—, —N(R^{e})—C(O)—, —C(O)—N(R^{e})—, —N(R^{e})C(O)N(R^{f})—, —C(O)—, —C(O)O—, —OC(O)—, —S—, —SO—, —SO₂—, —S(O)₂N(R^{e})—, —N(R^{e})SO₂—, wherein R^{e} and R^{f} are each independently selected from hydrogen or (1-2C)alkyl;

L is —(CR^{g}R^{h})ₚ—, wherein p is 3, 4 or 5, and R^{g} and R^{h} are at each occurrence independently selected from hydrogen, (1-2C)alkyl or (1-2C)haloalkyl, or R^{g} and R^{h} can be linked such that, together with the carbon atom to which they are attached, they form a (3-6C)cycloalkyl ring;

X² is selected from —O—, —N(Rⁱ)—, —N(Rⁱ)—C(O)—, —C(O)—N(Rⁱ)—, —N(R^{j})C(O)N(Rⁱ)—, —C(O)—, —C(O)O—, —OC(O)—, —S—, —SO—, —SO₂—, —S(O)₂N(Rⁱ)—, —N(Rⁱ)SO₂—, wherein Rⁱ and R^{j} are each independently selected from hydrogen or (1-2C)alkyl; and

Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;

or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)haloalkyl, (1-2C)alkoxy or (1-2C)haloalkoxy;
(24) R₃ is selected from hydroxyl, (1-2C)alkoxy, or a group of the formula:

—X¹-L-X²-Q

wherein:

X¹ is selected from —O—, —N(R^{e})—, —N(R^{e})—C(O)—, —C(O)—N(R^{e})—, —C(O)O—, —OC(O)—, —S—, —SO—, —SO₂—, —S(O)₂N(R^{e})—, —N(R^{e})SO₂—, wherein R^{e} is independently selected from hydrogen or (1-2C)alkyl;

L is —(CR^{g}R^{h})ₚ—, wherein p is 3, 4 or 5, and R^{g} and R^{h} are at each occurrence independently selected from hydrogen, (1-2C)alkyl or (1-2C)haloalkyl, or R^{g} and R^{h} can be linked such that, together with the carbon atom to which they are attached, they form a (3-6C)cycloalkyl ring;

X² is selected from —O—, —N(Rⁱ)—, —N(Rⁱ)—C(O)—, —C(O)—N(Rⁱ)—, —C(O)O—, —OC(O)—, —S—, —SO—, —SO₂—, —S(O)₂N(Rⁱ)—, —N(Rⁱ)SO₂—, wherein Rⁱ is independently selected from hydrogen or (1-2C)alkyl; and

Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;

or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)haloalkyl, (1-2C)alkoxy or (1-2C)haloalkoxy;
(25) R₃ is selected from hydroxyl, (1-2C)alkoxy, or a group of the formula:

—X¹-L-X²-Q

wherein:

X¹ is selected from —O— or —N(R^{e})—, wherein R^{e} is independently selected from hydrogen or (1-2C)alkyl;

L is —(CR^{g}R^{h})ₚ—, wherein p is 3, 4 or 5, and R^{g} and R^{h} are at each occurrence independently selected from hydrogen, (1-2C)alkyl or (1-2C)haloalkyl, or R^{g} and R^{h} can be linked such that, together with the carbon atom to which they are attached, they form a (3-4C)cycloalkyl ring;

X² is selected from —O—, —N(Rⁱ)—, —N(Rⁱ)—C(O)—, —C(O)—N(Rⁱ)—, —C(O)O—, —OC(O)—, —S—, —SO—, —SO₂—, —S(O)₂N(Rⁱ)—, —N(Rⁱ)SO₂—, wherein Rⁱ is independently selected from hydrogen or (1-2C)alkyl; and

Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;

or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)haloalkyl, (1-2C)alkoxy or (1-2C)haloalkoxy;
(26) R₃ is selected from hydroxyl, (1-2C)alkoxy, or a group of the formula:

—X¹-L-X²-Q

wherein:

X¹ is —O—;

L is —(CR^{g}R^{h})ₚ—, wherein p is 3 or 4, and R^{g} and R^{h} are at each occurrence independently selected from hydrogen or (1C)alkyl or (1C)haloalkyl;

X² is selected from —O— or —N(Rⁱ)—, wherein Rⁱ is independently selected from hydrogen or (1-2C)alkyl; and

Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;

or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)haloalkyl, (1-2C)alkoxy or (1-2C)haloalkoxy;
(27) R₃ is selected from hydroxyl, methoxy, or a group of the formula:

—X¹-L-X²-Q

wherein:

X¹ is —O—;

L is —(CR^{g}R^{h})ₚ—, wherein p is 3, and R^{g} and R^{h} are at each occurrence independently selected from hydrogen or methyl;

X² is selected from —O— or —N(Rⁱ)—, wherein Rⁱ is independently selected from hydrogen or (1-2C)alkyl; and

Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;

or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)haloalkyl, (1-2C)alkoxy or (1-2C)haloalkoxy;
(28) R₃ is selected from hydroxyl, methoxy, or a group of the formula:

—X¹-L-X²-Q

wherein:

X¹ is —O—;

L is —(CR^{g}R^{h})ₚ—, wherein p is 3, and R^{g} and R^{h} are at each occurrence independently selected from hydrogen or methyl;

X² is selected from —O— or —N(Rⁱ)—, wherein Rⁱ is independently selected from hydrogen or (1-2C)alkyl; and

Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;
(29) R₃ is selected from hydroxyl, methoxy, or a group of the formula:

—X¹-L-X²-Q

wherein:

X¹ is —O—;

L is —(CR^{g}R^{h})ₚ—, wherein p is 3, and R^{g} and R^{h} are at each occurrence independently selected from hydrogen or methyl;

X² is selected from —O— or —N(Rⁱ)—, wherein Rⁱ is independently selected from hydrogen or (1-2C)alkyl; and

Q is selected from hydrogen or methyl optionally substituted with one or more halo substituents;
(30) R₃ is selected from hydroxyl, methoxy, or a group of the formula:

—X¹-L-X²-Q

wherein:

X¹ is —O—;

L is —(CR^{g}R^{h})ₚ—, wherein p is 3, and R^{g} and R^{h} are at each occurrence independently selected from hydrogen or methyl;

X² is selected from —O— or —N(Rⁱ)—, wherein Rⁱ is independently selected from hydrogen or methyl; and

Q is selected from hydrogen or methyl;
(31) R₃ is selected from hydroxyl, methoxy, or a group of the formula:

—X¹-L-X²-Q

wherein:

X¹ is —O—;

L is —(CR^{g}R^{h})ₚ—, wherein p is 3, and R^{g} and R^{h} are at each occurrence independently selected from hydrogen or methyl;

X² is —O—; and

Q is as defined in any one of paragraphs (23) to (30) above;
(32) R₃ is a group of the formula:

—X¹-L-X²-Q

wherein:

X¹, L, X² and Q are as defined in any one of paragraphs (23) to (30) above;
(33) R₃ is a group of the formula:

—X¹-L-X²-Q

wherein:

X¹ is —O—;

L is —(CR^{g}R^{h})ₚ—, wherein p is 3, and R^{g} and R^{h} are at each occurrence independently selected from hydrogen or methyl;

X² is —O—; and

Q is as defined in any one of paragraphs (20) to (27) above;
(34) R₃ is group of the formula:

—X¹-L-X²-Q

wherein:

X¹ is selected from —O—, —N(R^{e})—, —N(R^{e})—C(O)—, —C(O)—N(R^{e})—, —N(R^{e})C(O)N(R^{f})—, —C(O)—, —C(O)O—, —OC(O)—, —S—, —SO—, —SO₂—, —S(O)₂N(R^{e})—, —N(R^{e})SO₂—, wherein R^{e} and R^{f} are each independently selected from hydrogen or (1-2C)alkyl;

L is —(CR^{g}R^{h})ₚ—, wherein p is 3, 4 or 5, and R^{g} and R^{h} are at each occurrence independently selected from hydrogen, (1-2C)alkyl or (1-2C)haloalkyl, or R^{g} and R^{h} can be linked such that, together with the carbon atom to which they are attached, they form a (3-6C)cycloalkyl ring;

X² is selected from —O—, —N(Rⁱ)—, —N(Rⁱ)—C(O)—, —C(O)—N(Rⁱ)—, —N(R^{j})C(O)N(R)—, —C(O)—, —C(O)O—, —OC(O)—, —S—, —SO—, —SO₂—, —S(O)₂N(R)—, —N(Rⁱ)SO₂—, wherein Rⁱ and R^{j} are each independently selected from hydrogen or (1-2C)alkyl; and

Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;

or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)haloalkyl, (1-2C)alkoxy or (1-2C)haloalkoxy;
(35) R₃ is a group of the formula:

—X¹-L-X²-Q

wherein:

X¹ is selected from —O—, —N(R^{e})—, —N(R^{e})—C(O)—, —C(O)—N(R^{e})—, —C(O)O—, —OC(O)—, —S—, —SO—, —SO₂—, —S(O)₂N(R^{e})—, —N(R^{e})SO₂—, wherein R^{e} is independently selected from hydrogen or (1-2C)alkyl;

L is —(CR^{g}R^{h})ₚ—, wherein p is 3, 4 or 5, and R^{g} and R^{h} are at each occurrence independently selected from hydrogen, (1-2C)alkyl or (1-2C)haloalkyl, or R^{g} and R^{h} can be linked such that, together with the carbon atom to which they are attached, they form a (3-6C)cycloalkyl ring;

X² is selected from —O—, —N(Rⁱ)—, —N(Rⁱ)—C(O)—, —C(O)—N(Rⁱ)—, —C(O)—, —OC(O)—, —S—, —SO—, —SO₂—, —S(O)₂N(Rⁱ)—, —N(Rⁱ)SO₂—, wherein Rⁱ is independently selected from hydrogen or (1-2C)alkyl; and

Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;

or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)haloalkyl, (1-2C)alkoxy or (1-2C)haloalkoxy;
(36) R₃ is a group of the formula:

—X¹-L-X²-Q

wherein:

X¹ is selected from —O— or —N(R^{e})—, wherein R^{e} is independently selected from hydrogen or (1-2C)alkyl;

L is —(CR^{g}R^{h})ₚ—, wherein p is 3, 4 or 5, and R^{g} and R^{h} are at each occurrence independently selected from hydrogen, (1-2C)alkyl or (1-2C)haloalkyl, or R^{g} and R^{h} can be linked such that, together with the carbon atom to which they are attached, they form a (3-4C)cycloalkyl ring;

X² is selected from —O—, —N(Rⁱ)—, —N(Rⁱ)—C(O)—, —C(O)—N(Rⁱ)—, —C(O)O—, —OC(O)—, —S—, —SO—, —SO₂—, —S(O)2N(R′)-, -N(R)S02-, wherein Rⁱ is independently selected from hydrogen or (1-2C)alkyl; and

Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;

or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)haloalkyl, (1-2C)alkoxy or (1-2C)haloalkoxy;
(37) R₃ is a group of the formula:

—X¹-L-X²-Q

wherein:

X¹ is —O—;

L is —(CR^{g}R^{h})ₚ—, wherein p is 3 or 4, and R^{g} and R^{h} are at each occurrence independently selected from hydrogen or (1C)alkyl or (1C)haloalkyl;

X² is selected from —O— or —N(Rⁱ)—, wherein Rⁱ is independently selected from hydrogen or (1-2C)alkyl; and

Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;

or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)haloalkyl, (1-2C)alkoxy or (1-2C)haloalkoxy;
(38) R₃ is a group of the formula:

—X¹-L-X²-Q

wherein:

X¹ is —O—;

L is —(CR^{g}R^{h})ₚ—, wherein p is 3, and R^{g} and R^{h} are at each occurrence independently selected from hydrogen or methyl;

X² is selected from —O— or —N(Rⁱ)—, wherein Rⁱ is independently selected from hydrogen or (1-2C)alkyl; and

Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;

or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)haloalkyl, (1-2C)alkoxy or (1-2C)haloalkoxy;
(39) R₃ is a group of the formula:

—X¹-L-X²-Q

wherein:

X¹ is —O—;

L is —(CR^{g}R^{h})ₚ—, wherein p is 3, and R^{g} and R^{h} are at each occurrence independently selected from hydrogen or methyl;

X² is selected from —O— or —N(Rⁱ)—, wherein Rⁱ is independently selected from hydrogen or (1-2C)alkyl; and

Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;
(40) R₃ is a group of the formula:

—X¹-L-X²-Q

wherein:

—X¹ is —O—;

L is —(CR^{g}R^{h})ₚ—, wherein p is 3, and R^{g} and R^{h} are at each occurrence independently selected from hydrogen or methyl;

X² is selected from —O— or —N(Rⁱ)—, wherein Rⁱ is independently selected from hydrogen or (1-2C)alkyl; and

Q is selected from hydrogen or methyl optionally substituted with one or more halo substituents;
(41) R₃ is a group of the formula:

—X¹-L-X²-Q

wherein:

X¹ is —O—;

L is —(CR^{g}R^{h})ₚ—, wherein p is 3, and R^{g} and R^{h} are at each occurrence independently selected from hydrogen or methyl;

X² is selected from —O— or —N(Rⁱ)—, wherein Rⁱ is independently selected from hydrogen or methyl; and

Q is selected from hydrogen or methyl;
(42) R₃ is a group of the formula:

—X¹-L-X²-Q

wherein:

X¹ is —O—;

L is —(CR^{g}R^{h})ₚ—, wherein p is 3, and R^{g} and R^{h} are at each occurrence independently selected from hydrogen or methyl;

X² is —O—; and

Q is hydrogen or methyl.

Suitably, m is as defined in any one of paragraphs (1) to (4) above. In an embodiment, m is as defined in any one of paragraphs (1) to (3) above.

Suitably, when m is 1 or 2, the R₁ group or groups present are in the ortho and para positions (relative to the point of attachment to the —CH═CH— group).

Suitably, when m is 1, the RI group is in the ortho and para position (relative to the point of attachment to the —CH═CH— group). In an embodiment, m is 1 and R₁ is a group as defined herein which is present in the ortho position (relative to the point of attachment to the —CH═CH— group). In another embodiment, m is 1 and R₁ is a group as defined herein which is present in the para position (relative to the point of attachment to the —CH═CH— group).

Suitably, R₁ is a substituent group as defined in any one of paragraphs (5) to (11) above. In an embodiment, R₁ is as defined in any one of paragraphs (7), (8) or (9) above. In another embodiment, RI is as defined in paragraphs (10) or (11) above.

Suitably, n is as defined in any one of paragraphs (12) to (15) above. In an embodiment, n is as defined in any one of paragraphs (12) to (14) above.

Suitably, when n is 1, the R₂ group is in the ortho or meta position (relative to the point of attachment to the —CH═CH— group). In an embodiment, n is 1 and R2 is a group as defined herein which is present in the ortho position (relative to the point of attachment to the —CH═CH— group). In another embodiment, n is 1 and R2 is a group as defined herein which is present in the meta position (relative to the point of attachment to the —CH═CH— group).

Suitably, R₂ is as defined in any one of paragraphs (16) to (22) above. In an embodiment, R₂ is as defined in any one of paragraphs (18), (19) or (20) above. In a further embodiment, R2 is as defined in paragraph (21) or (22) above.

Suitably, R₃ is as defined in any one of paragraphs (23) to (33) above. In an embodiment, Q is as defined in any one of paragraphs (29) to (33) above. In a particular embodiment, Q is as defined in paragraph (32) or (33) above.

More suitably, R₃ is as defined in any one of paragraphs (34) to (42) above. In an embodiment, Q is as defined in any one of paragraphs (39) to (42) above. In a particular embodiment, Q is as defined in paragraph (40), (41) or (42) above.

In certain embodiments, p is 3 or 4. In a particular embodiment, p is 3.

In a particular group of compounds, the compounds have the structural formula I wherein n is 0, i.e. the compounds have the structural formula (IA):

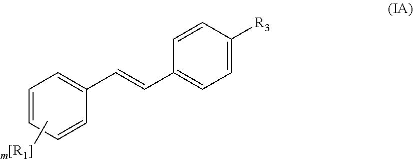

wherein

m, R1 and R₃ each have any one of the defintions set out herein.

In a further group of compounds of formula (IA), m is 0 or 1 and any R1 substituent group present is in the ortho or para position (relative to the point of attachment to the —CH═CH— group).

In a further group of compounds of formula (IA), R₃ is as defined in any one of paragraphs (34) to (42) above and m and R₁ each have any one of the defintions set out herein. In a further group of compounds of formula (IA), R₃ is as defined paragraph (41) or (42) above and m and R₁ each have any one of the defintions set out herein.

Particular compounds of the present invention for use in the treatment of a disease or condition as defined herein (e.g. hypercholesterolemia) include any one of the following:

(E)-N,N-Dimethyl-3-(4-styrylphenoxy)propan-1-amine; 15a
(E)-1-((4-Methylpentyl)oxy)-4-styrylbenzene; 39
(E)-1-(3-Methoxypropoxy)-4-styrylbenzene; 41
(E)-3-(4-(4-Methoxystyryl)phenoxy)-N,N-dimethylpropan-1-amine; 44a
(E)-3-(4-(3-Methoxystyryl)phenoxy)-N,N-dimethylpropan-1-amine; 44b
(E)-3-(4-(2-Methoxystyryl)phenoxy)-N,N-dimethylpropan-1-amine; 44c
(E)-N,N-Dimethyl-3-(4-(2-(naphthalen-2-yl)vinyl)phenoxy) propan-1-amine; 45
(E)-3-(4-(4-Fluorostyryl)phenoxy)-N,N-dimethylpropan-1-amine; 46a
(E)-3-(4-(3-Fluorostyryl)phenoxy)-N,N-dimethylpropan-1-amine; 46b
(E)-N,N-Dimethyl-3-(4-(4-nitrostyryl)phenoxy)propan-1-amine; 47a
(E)-N,N-Dimethyl-3-(4-(3-nitrostyryl)phenoxy)propan-1-amine; 47b
(E)-N,N-Dimethyl-3-(4-(2-nitrostyryl)phenoxy)propan-1-amine; 47c
(E)-3-(2-Fluoro-4-styrylphenoxy)-N,N-dimethylpropan-1-amine; 62
(E)-N-Methyl-3-(4-styrylphenoxy)propan-1-amine; 69

### (E)-4-Styrylphenol; 32⁵⁶

### (E)-3-(4-Styrylphenoxy)propan-1-ol; 40⁵⁷

(E)-1-Methoxy-4-styrylbenzene; 65⁵⁸
or a pharmaceutically acceptable salt or solvate thereof.

Particular compounds of the present invention are: (E)-N,N-Dimethyl-3-(4-styrylphenoxy)propan-1-amine; 15a and (E)-3-(4-Styrylphenoxy)propan-1-ol; 40.

A particular compound for use in therapy as defined herein is (E)-3-(4-Styrylphenoxy)propan-1-ol.

### Novel Compounds

In another aspect, the present invention provides a compound of Formula I as defined hereinbefore, with the proviso that the compound is not one of the following:

(E)-N,N-Dimethyl-3-(4-styrylphenoxy)propan-1-amine; 15a

### (E)-4-Styrylphenol; 32

### (E)-3-(4-Styrylphenoxy)propan-1-ol; 40

(E)-1-Methoxy-4-styrylbenzene; 65
(E)-N,N-Diethyl-2-(4-styrylphenoxy)propan-1-amine.

In another aspect, the present invention provides a compound of formula I as defined hereinbefore wherein R₃ is a group of the formula:

—X¹-L-X²-Q

wherein X¹, L, X² and Q are each as defined hereinbefore, with the proviso that:

(i) Q and Rⁱ are not both methyl or ethyl when X¹ is —O—, p is 3, R^{g} and R^{h} are both hydrogen, X² is —N(Rⁱ)—, and m and n are both 0; and

(ii) Q is not hydrogen when X¹ and X² are —O—.

In another aspect, the present invention provides a compound of formula I as defined hereinbefore wherein R₃ is a group of the formula:

—X¹-L-X²-Q

wherein X¹, L, X² and Q are each as defined hereinbefore, with the proviso that:

(i) Q and Rⁱ are both hydrogen or methyl when X¹ is —O—, p is 3, R^{g} and R^{h} are both hydrogen; and X² is —N(Rⁱ)—; and

(ii) Q is not hydrogen when X¹ and X² is —O—.

In an embodiment, Q is methyl and Rⁱ is hydrogen or methyl.

In another aspect, the present invention provides a compound of formula I as defined hereinbefore wherein R₃ is a group of the formula:

—X¹-L-X²-Q

wherein m, R₁, n, R₂, X¹, L, X² and Q are each as defined hereinbefore, with the proviso that m and n are not both 0. Suitably, R₃ is as defined in any one of paragraphs (34) to (42) above. In an embodiment, Q is as defined in any one of paragraphs (39) to (42) above. In a particular embodiment, Q is as defined in paragraph (40), (41) or (42) above. In a particular groups of compounds, m is 1 or 2 and n is 0 or 1.

Particular novel compounds of the present invention include any one of the following:

(E)-1-((4-Methylpentyl)oxy)-4-styrylbenzene; 39
(E)-1-(3-Methoxypropoxy)-4-styrylbenzene; 41
(E)-3-(4-(4-Methoxystyryl)phenoxy)-N,N-dimethylpropan-1-amine; 44a
(E)-3-(4-(3-Methoxystyryl)phenoxy)-N,N-dimethylpropan-1-amine; 44b
(E)-3-(4-(2-Methoxystyryl)phenoxy)-N,N-dimethylpropan-1-amine; 44c
(E)-N,N-Dimethyl-3-(4-(2-(naphthalen-2-yl)vinyl)phenoxy) propan-1-amine; 45
(E)-3-(4-(4-Fluorostyryl)phenoxy)-N,N-dimethylpropan-1-amine; 46a
(E)-3-(4-(3-Fluorostyryl)phenoxy)-N,N-dimethylpropan-1-amine; 46b
(E)-N,N-Dimethyl-3-(4-(4-nitrostyryl)phenoxy)propan-1-amine; 47a
(E)-N,N-Dimethyl-3-(4-(3-nitrostyryl)phenoxy)propan-1-amine; 47b
(E)-N,N-Dimethyl-3-(4-(2-nitrostyryl)phenoxy)propan-1-amine; 47c
(E)-3-(2-Fluoro-4-styrylphenoxy)-N,N-dimethylpropan-1-amine; 62
(E)-N-Methyl-3-(4-styrylphenoxy)propan-1-amine; 69
or a pharmaceutically acceptable salt or solvate thereof.

The various functional groups and substituents making up the compounds of the present invention are typically chosen such that the molecular weight of the compound does not exceed 1000. More usually, the molecular weight of the compound will be less than 750, for example less than 700, or less than 650, or less than 600, or less than 550. More preferably, the molecular weight is less than 525 and, for example, is 500 or less.

Suitable or preferred features of any compounds of the present invention may also be suitable features of any other aspect.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess the desired pharmacological activity.

The present invention also encompasses compounds of the invention as defined herein which comprise one or more isotopic substitutions. For example, H may be in any isotopic form, including ¹H, ²H (D) and ³H (T); C may be in any isotopic form including ¹²C, ¹³C, and ¹⁴C; and O may be in any isotopic form, including ¹⁶O and ¹⁸P; and the like.

It is also to be understood that certain compounds of the invention may exist in solvated as well as unsolvated forms such as, for example, hydrated forms. It is to be understood that the invention encompasses all such solvated forms that possess the desired pharmacological activity.

It is also to be understood that certain compounds of the invention may exhibit polymorphism, and that the invention encompasses all such forms that possess the desired pharmacological activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

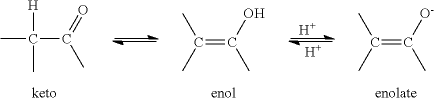

Compounds of the invention containing an amine function may also form N-oxides. A reference herein to a compound of the formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

The compounds of the invention may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the invention and in-vivo cleavable amide derivatives that may be formed at a carboxy group or an amino group in a compound of the invention.

Accordingly, the present invention includes those compounds of the formula I as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human or animal body by way of metabolism of a precursor compound, that is a compound of the formula I may be a synthetically-produced compound or a metabolically-produced compound.

A suitable pharmaceutically acceptable pro-drug of a compound of the formula I is one that is based on reasonable medical judgement as being suitable for administration to the human or animal body without undesirable pharmacological activities and without undue toxicity.

Various forms of pro-drug have been described, for example in the following documents :

a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);

### d) H. Bundgaard, Advanced Drug Delivery Reviews, 8, 1-38 (1992);

### e) H. Bundgaard, et al., Journal of Pharmaceutical Sciences, 77, 285 (1988);

f) N. Kakeya, et al., Chem. Pharm. Bull., 32, 692 (1984);

### g) T. Higuchi and V. Stella, “Pro-Drugs as Novel Delivery Systems”, A.C.S. Symposium Series, Volume 14; and

h) E. Roche (editor), “Bioreversible Carriers in Drug Design”, Pergamon Press, 1987.

A suitable pharmaceutically acceptable pro-drug of a compound of the formula I that possesses a carboxy group is, for example, an in vivo cleavable ester thereof. An in vivo cleavable ester of a compound of the formula I containing a carboxy group is, for example, a pharmaceutically acceptable ester which is cleaved in the human or animal body to produce the parent acid. Suitable pharmaceutically acceptable esters for carboxy include C₁₋₆alkyl esters such as methyl, ethyl and tert-butyl, C₁₋₆alkoxymethyl esters such as methoxymethyl esters, C₁₋₆alkanoyloxymethyl esters such as pivaloyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆alkyl esters such as methoxycarbonyloxymethyl and 1-methoxycarbonyloxyethyl esters.

A suitable pharmaceutically acceptable pro-drug of a compound of the formula I that possesses a hydroxy group is, for example, an in vivo cleavable ester or ether thereof. An in vivo cleavable ester or ether of a compound of the formula I containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀ loalkanoyl groups such as acetyl, benzoyl, phenylacetyl and substituted benzoyl and phenylacetyl groups, C₁₋₁₀ alkoxycarbonyl groups such as ethoxycarbonyl, N,N-(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl)piperazin-1-ylmethyl. Suitable pharmaceutically acceptable ether forming groups for a hydroxy group include a-acyloxyalkyl groups such as acetoxymethyl and pivaloyloxymethyl groups.

A suitable pharmaceutically acceptable pro-drug of a compound of the formula I that possesses a carboxy group is, for example, an in vivo cleavable amide thereof, for example an amide formed with an amine such as ammonia, a C₁₋₄ alkylamine such as methylamine, a (C₁₋₄alkyl)₂amine such as dimethylamine, N-ethyl-N-methylamine or diethylamine, a C₁₋₄ alkoxy-C₂₋₄alkylamine such as 2-methoxyethylamine, a phenyl-C₁₋₄alkylamine such as benzylamine and amino acids such as glycine or an ester thereof.

A suitable pharmaceutically acceptable pro-drug of a compound of the formula I that possesses an amino group is, for example, an in vivo cleavable amide derivative thereof. Suitable pharmaceutically acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the formula I may be exerted in part by one or more metabolites that are formed within the human or animal body after administration of a compound of the formula I. As stated hereinbefore, the in vivo effects of a compound of the formula I may also be exerted by way of metabolism of a precursor compound (a pro-drug).

It shall also be appreciated that compounds of the formula I may also be covalently linked (at any suitable position) to other groups such as, for example, solubilising moieties (for example, PEG polymers), moieties that enable them to be bound to a solid support (such as, for example, biotin-containing moieties), and targeting ligands (such as antibodies or antibody fragments).

### Synthesis

In the description of the synthetic methods described below in the accompanying example section and in the referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃.OEt₂. A suitable alternative protecting group for a primary amino group is, for example, a phthaloyl group which may be removed by treatment with an alkylamine, for example dimethylaminopropylamine, or with hydrazine.

The person skilled in the art will recognise that the compounds of the invention may be prepared, in known manner, in a variety of ways. Compounds of formula I can be prepared by the methods given below, by the methods given in the experimental or by analogous methods. The routes described are merely illustrative of some of the methods that can be employed for the synthesis of compounds of formulae I and the person skilled in the art will appreciate that the order of the reaction steps is not limited to those described. It will also be appreciated that the assignment of nucleophile and electrophile is not limited to that described herein and in some cases it may be appropriate for the assignment to be reversed. Different approaches to synthetic chemistry strategy are described in “Organic Synthesis: The Disconnection Approach”, 2^{nd} edition, S. Warren and P. Wyatt (2008).

### Pharmaceutical Compositions

According to a further aspect of the invention there is provided a pharmaceutical composition which comprises a compound of the invention as defined hereinbefore, or a pharmaceutically acceptable salt or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of proliferative disease is an amount sufficient to symptomatically relieve in a warm-blooded animal, particularly a human the symptoms of infection, to slow the progression of infection, or to reduce in patients with symptoms of infection the risk of getting worse.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

It is understood that the compounds of the invention are inhibitors of squalene synthase. As a consequence, they are potentially useful therapeutic agents for the treatment of dieases or conditions in which squalene synthase activity is implicated.

Thus, in one aspect, the present invention relates to a compound of the invention as defined herein, or a pharmaceutically acceptable salt or solvate thereof, or a pharmaceutical composition as defined herein, for use in therapy.

In another aspect, the present invention relates to a compound of the invention as defined herein, or a pharmaceutically acceptable salt or solvate thereof, or a pharmaceutical composition as defined herein, for use in the treatment of diseases or conditions in which squalene synthase activity is implicated.

In another aspect, the present invention relates to the use of a compound of the invention as defined herein, or a pharmaceutically acceptable salt or solvate thereof, in the manufacture of a medicament for use in the treatment of diseases or conditions in which squalene synthase activity is implicated.

In another aspect, the present invention relates to a method of treating a disease or condition in which squalene synthase activity is implicated, said method comprising administering to a subject in need of such treatment a therapeutically effective amount of a compound of the invention as defined herein, or a pharmaceutically acceptable salt or solvate thereof, or a pharmaceutical composition as defined herein.

Examples of conditions in which squalene synthase activity is implicated include hyperlipidaemia and, in particular, hypercholesterolaemia, diseases or conditions associated with hyperlipidaemia/hypercholesterolaemia (e.g. atherosclerosis (i.e. atherosclerotic blood lesions) and theft secondary diseases, for example, coronary arterial diseases, cerebral ischemia, intermittent claudication and gangrene) and trypansomal diseases (such as sleeping sickness and chagas disease).

In another aspect, the present invention provides a compound, or a pharmaceutically acceptable salt or solvate thereof, or a pharmaceutical composition as defined herein, for use in the treatment of hypercholesterolaemia.

In another aspect, the present invention provides the use of a compound, or a pharmaceutically acceptable salt or solvate thereof, in the manufacture of a medicament for use in the treatment of hypercholesterolaemia.

In another aspect, the present invention provides a method of treating hypercholesterolaemia, said method comprising administering to a subject in need of such treatment a therapeutically effective amount of a compound, or a pharmaceutically acceptable salt or solvate thereof, or a pharmaceutical composition as defined herein.

In another aspect, the present invention provides a compound, or a pharmaceutically acceptable salt or solvate thereof, or a pharmaceutical composition as defined herein, for use in the treatment of diseases or conditions associated with hypercholesterolamia.

As previously indicated, examples of such conditions that are associated with hypercholesterolaemia include atherosclerosis (i.e. atherosclerotic blood lesions) and their secondary diseases, for example, coronary arterial diseases, cerebral ischemia, intermittent claudication and gangrene.

In another aspect, the present invention provides the use of a compound, or a pharmaceutically acceptable salt or solvate thereof, in the manufacture of a medicament for use in the treatment of for use in the treatment of diseases or conditions associated with hypercholesterolamia.

In another aspect, the present invention provides a method of treating diseases or conditions associated with hypercholesterolemia, said method comprising administering to a subject in need of such treatment a therapeutically effective amount of a compound, or a pharmaceutically acceptable salt or solvate thereof, or a pharmaceutical composition as defined herein.

In another aspect, the present invention provides a compound, or a pharmaceutically acceptable salt or solvate thereof, or a pharmaceutical composition as defined herein, for use in the production of a squalene synthase inhibitory effect.

In another aspect, the present invention provides the use of a compound, or a pharmaceutically acceptable salt or solvate thereof, in the manufacture of a medicament for use in the production of a squalene synthase effect.

In another aspect, the present invention provides a method of producing a squalene synthase inhibitory effect in vitro, said method comprising administering an effective amount of a compound, or a pharmaceutically acceptable salt or solvate thereof.

In another aspect, the present invention provides a method of producing a squalene synthase inhibitory effect in vivo, said method comprising administering an effective amount of a compound, or a pharmaceutically acceptable salt or solvate thereof.

### Routes of Administration

The compounds of the invention or pharmaceutical composition comprising the active compound may be administered to a subject by any convenient route of administration, whether systemically/peripherally or topically (i.e. at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The compounds of the invention may be administered alone as a monotherapy or may administered in combination with one or more additional therapeutic agents or with an appropriate dietary regime. The selection of the one or more additional therapeutic agents will of course vary depending on the disease or condition to be treated and its severity.

It is commonplace to use combination therapies to treat certain medical conditions.

Therefore, the treatment defined hereinbefore may be applied as a sole therapy or may involve, in addition to the compound of the invention, treatment with one or more additional therapeutic agents.

Such conjoint/combination treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to a particular aspect of the invention there is provided a combination for use in the treatment of a disease or condition as defined hereinbefore, comprising a compound of the invention as defined hereinbefore, or a pharmaceutically acceptable salt or solvate thereof, and another therapeutic agent.

According to this aspect of the invention there is provided a combination for use in the treatment of a disease or condition as defined hereinbefore, the combination comprising a compound of the invention as defined hereinbefore, or a pharmaceutically acceptable salt or solvate thereof, and one or more additional therapeutic agents.

In a further aspect of the invention there is provided a compound of the invention or a pharmaceutically acceptable salt or solvate thereof, in combination with one or more additional therapeutic agents.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt or solvate thereof in combination with one or more additional therapeutic agents in association with a pharmaceutically acceptable diluent or carrier.

According to a particular aspect of the invention there is provided a combination suitable for use in the treatment of a disease or condition as defined hereinbefore comprising a compound of the invention as defined hereinbefore, or a pharmaceutically acceptable salt or solvate thereof, and another therapeutic agent.

Examples of the one or more additional therapeutic agents include other lipid/cholesterol lowering agents, such as HMG-CoA reductase inhibitors (e.g. atorvastatin, pravastatin, fluvastatin, simvastatin, lovostatin and rosuvastatin), bile acid sequestrants, ezetimibe, fibrates, niacin or ω-3 fatty acids.

In an embodiment, the one or more additional therapeutic agents is a HMG-CoA reductase inhibitors (e.g. atorvastatin, pravastatin, fluvastatin, simvastatin and rosuvastatin).

### EXAMPLES

### Synthesis of Compounds

### General Experimental

All reactions involving organometallic or other moisture-sensitive reagents were carried out under a nitrogen or argon atmosphere using standard vacuum line techniques and glassware that was oven dried and cooled under nitrogen before use. Solvents were dried according to the procedure outlined by Grubbs et al. (Organometallics, 1996, 15, 1518). Water was purified by an Elix® UV-10 system. All other reagents were used as supplied (analytical or HPLC grade) without prior purification. Thin layer chromatography was performed on aluminium plates coated with 60 F₂₅₄ silica. Plates were visualised using UV light (254 nm), or 1% aq KMnO₄. Flash column chromatography was performed on Kieselgel 60 silica. Melting points were recorded on a Gallenkamp Hot Stage apparatus and are uncorrected. IR spectra were recorded on a Bruker Tensor 27 FT-IR spectrometer with a diamond ATR module. Selected characteristic peaks are reported in cm⁻¹. NMR spectra were recorded on Bruker Avance spectrometers at rt in a solution of deuterated acetone unless stated otherwise. The field was locked by external referencing to the relevant deuteron resonance. Chemical shifts (8) are reported in ppm and coupling constants J in Hz. Low resolution mass spectra were recorded on either a VG MassLab 20-250 or a Micromass Platform 1 spectrometer. Accurate mass measurements were run on either a Bruker MicroTOF internally calibrated with polyalanine, or a Micromass GCT instrument fitted with a Scientific Glass Instruments BPX5 column (15 m×0.25 mm) using amyl acetate as a lock mass.

### General Procedures

### General Procedure 1-Alkylation of Hydroxides

K₂CO₃ (3 eq.) was added to a solution of the requisite alcohol (1 eq.) in DMF (1 mL) in a microwave vial. The resulting suspension was heated at 90° C. for 30 min before addition of a solution of the alkyl chloride (1.1 eq.), neutralised previously with K₂CO₃, in DMF (1 mL). The microwave vial was sealed and heated at 90° C. for 16 h. After cooling, the reaction mixture was quenched with H₂O (10 mL), extracted with EtOAc (3×15 mL), the organic layer washed with brine, dried (Na₂SO₄), filtered and concentrated in vacuo to yield the crude product. Purification by column chromatography afforded the desired product.

General Procedure 2-Preparation of Stilbenes by Heck Coupling using Pd(OAc)₂

Iodoarene (1 eq.), requisite styrene (1.2 eq.), Et₃N (2.5 eq.), Pd(OAc)₂ (0.03 eq.) and PPh₃ (0.06 eq.) were dissolved in degassed 1,4-dioxane (2 mL) in a sealed microwave vial, and heated to 100° C. for 16 h. The reaction mixture was allowed to cool, then quenched 1M HCl_{(aq)} (100 mL) and extracted with EtOAc (3×30 mL); the organic phase was washed with water, dried (Na₂SO₄), filtered and concentrated in vacuo to yield the crude product. Purification by column chromatography afforded the desired product.

General Procedure 3-Preparation of Stilbenes by Heck Coupling using Pd(NH₃)₂Cl₂

Haloarene (1 eq.), requisite styrene (1.5 eq.), n-Bu₃N (2 eq.), Pd(NH₃)2Cl₂ (0.015 eq.) and tetrabutylammonium bromide (1 eq.) were dissolved in H₂O (6 mL) in a sealed microwave vial, and heated to 140° C. for 24 h. After cooling, the reaction mixture was extracted with EtOAc (3×15 mL), the organic phase dried (Na₂SO₄), filtered and concentrated in vacuo to yield the crude product. Purification by column chromatography afforded the desired product.

### General Procedure 4-Preparation of Substituted Styrenes

The requisite aldehyde (1 eq.) in 1,4-dioxane (5 mL) was added to K200₃ (1.6 eq.) and methyltriphenylphosphonium bromide (1.4 eq.), and heated to 100° C. for 16 h in a sealed microwave vial. After cooling, the reaction mixture was filtered, and volatiles were removed in vacuo to yield the crude product. The residue was then dissolved in hot n-pentane, cooled to 0° C., filtered and washed with cold n-pentane. The filtrate was dried (Na₂SO₄), filtered and concentrated in vacuo to give the desired product.

### General Procedure 5-PMB Protection of Alcohols

K₂CO₃ (3 eq.) was added to a stirred solution of the appropriate hydroxybenzaldehyde (1 eq.) and 1-(chloromethyl)-4-methoxybenzene (1.3 eq.) in DMF (3 mL) at 0° C., then stirred for 21 h at RT. The reaction mixture was then quenched and extracted with toluene. The organic layers were washed with brine, dried (Na₂SO₄), filtered and concentrated in vacuo to give the crude product. Purification by column chromatography (EtOAc in Pet Ether) gave the desired product.

General Procedure 6-Preparation of Stilbenes by Heck Coupling Using Pd(OAc)₂

Pd(OAc)₂ (0.05 eq.) and PPh₃ (0.15 eq.) were added to a sealed microwave vial with DMF (1 mL), and the resulting solution was flushed with nitrogen. The resulting orange solution was heated at 110° C. for 10 min, to give a black solution. A solution of appropriate styrene (1.3 eq.), iodoarene (1 eq.) and K200₃ (4 eq.) in DMF (0.5 mL) was then added, and the reaction mixture heated at 110° C. for a further 2 h. The reaction mixture was allowed to cool, and extracted with EtOAc. The combined organic layers were washed with brine, dried (Na₂SO₄), filtered and reduced in vacuo. Purification by column chromatography gave the desired product.

### Preparation of Intermediates

### 4-(3-(Dimethylamino)propoxy)benzaldehyde 17a⁵⁹

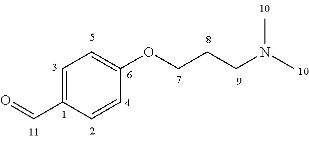

K₂CO₃ (999 mg, 7.23 mmol) was added to a solution of the 4-hydroxybenzaldehyde (419 mg, 2.65 mmol) in DMF (2 mL) in a microwave vial. The resulting suspension was heated at 90° C. for 30 min before addition of a solution of 3-dimethylamino-1-propylchloride hydrochloride (419 mg, 2.65 mmol), neutralised previously with K₂CO₃. The microwave vial was sealed and heated at 90° C. for 16 h. After cooling, the reaction mixture was quenched with H₂O (10 mL), extracted with EtOAc (3×15 mL), the organic layer washed with brine, dried (Na₂SO₄) and concentrated in vacuo to yield the crude product. Purification by column chromatography (8% Me0H, 2% Et₃N in CH2012) afforded the title compound as a brown viscous oil (173 mg, 32%). The analytical data were in accordance with those reported in the lit.⁵⁹ δ_{H} (400 MHz) 9.89 (1H, s, H11), 7.87 (2H, apparent ddd J 8.8, 2.7, 1.9, H2, H3), 7.11 (2H, apparent ddd J 8.8, 2.7, 1.9, H4, H5), 4.17 (2H, t J 6.4, H7), 2.44 (2H, t J 7.0, H9), 2.20 (6H, s, H10), 1.96 (2H, m, H8); δ_{C} (100 MHz) 191.3 (C11), 165.1 (C6), 132.6 (C2, C3), 131.1 (C1), 115.7 (C4, C5), 67.4 (C7), 56.6 (C9), 45.7 (010), 28.0 (C8); m/z (ESL⁺) 208 ([M+H]⁺).

### 1-Methoxy-4-vinylbenzene 30a⁶³

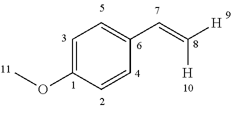

Following general procedure 4, 4-methoxybenzaldehyde (0.45 mL, 3.73 mmol) gave the title compound as a clear oil (458 mg, 90%). The analytical data were in accordance with those reported in the lit.⁶³ δ_{H} (400 MHz) 7.39 (2H, m, H4, H5), 6.90 (2H, apparent ddd J 8.8, 2.9, 2.1, H2, H3), 6.68 (1 H, dd J 17.6, 10.9, H7), 5.65 (1 H, dd J 17.6, 1.0, H10), 5.08 (1H, dd J 10.9, 1.0, H9), 3.79 (3H, s, H11); O_{c} (100 MHz) 160.6 (C1), 137.4 (C7), 131.2 (C6), 128.3 (C4, C5), 114.8 (C2, C3), 111.7 (C8), 55.6 (C11); HRMS (FI) C₉H₁₀O (M) requires 134.0732, found 134.0727.

### 1-Methoxy-3-vinylbenzene 30b⁶⁴

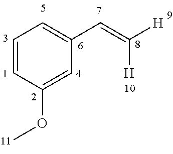

Following general procedure 4, 3-methoxybenzaldehyde (0.45 mL, 3.73 mmol) gave the title compound as a clear oil (361 mg, 72%). The analytical data were in accordance with those reported in the lit.⁶⁴ δ_{H} (400 MHz) 7.25 (1 H, m, arom.), 7.03 (2H, m, arom.), 6.84 (1H, m, arom.), 6.73 (1H, dd J 17.7, 10.9, H7), 5.81 (1H, dd J 17.7 , 0.9, H10), 5.23 (1H, dd J 10.9, 0.9, H9), 3.80 (3H, s, H11); δ_{C} (100 MHz) 161.0 (C2), 140.0 (C6), 137.9, 130.4, 119.6, 114.5, 114.5, 112.3, 55.5 (C11); HRMS (FI) C₉H₁₀O (M) requires 134.0732, found 134.0732.

### 1-Methoxy-2-vinylbenzene 30c⁶⁵

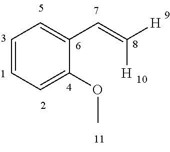

Following general procedure 4, 2-methoxybenzaldehyde (0.45 mL, 3.73 mmol) gave the title compound as a clear oil (214 mg, 43%). The analytical data were in accordance with those reported in the lit.⁶⁵ δ_{H} (400 MHz) 7.51 (1 H, dd J 7.6, 1.7, arom.), 7.25 (1H, m, arom.), 7.03 (1H, dd J 17.9, 11.3, H7), 6.94 (2H, m, arom.), 5.76 (1H, dd J 17.6, 1.7, H10), 5.21 (1H, dd J 11.2, 1.6, H9) 3.84 (3H, s, H11); δ_{C} (100 MHz) 157.8 (04), 132.6, 130.0, 127.2, 127.1, 121.4, 114.4, 112.0, 55.8 (C11); HRMS (FI) C₉H₁₀O (M) requires 134.0732, found 134.0729.

### (E)-2-Fluoro-4-styrylphenol 61⁶⁹

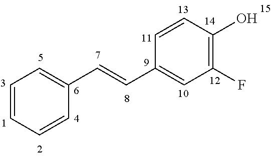 [SMILES: Oc1ccc(/C=C/c2ccccc2)cc1F]

Following general procedure 3, 4-bromo-2-fluorophenol (134 mg, 0.7 mmol) and styrene (0.12 mL, 1.05 mmol) gave the title compound as a cream solid (111 mg, 74%). The analytical data were in accordance with those reported in the lit.⁶⁹ δ_{H} (400 MHz) 8.84 (1H, br. s, H15), 7.55 (2H, d J 7.9, arom.), 7.40 (1H, dd J 12.7, 2.1, H10), 7.35 (2H, m, arom.), 7.24 (2H, m, arom.), 7.16 (1H, d J 16.4, H8), 7.10 (1H, d J 16.4, H7), 7.01 (1H, m, arom.); δ_{C} (100 MHz) 152.6 (d J239.9, C12), 145.4 (d J13.2, C14), 138.4, 131.1 (d J 6.6), 129.5, 128.3 (d J 2.2), 128.2, 128.1, 127.2, 124.2 (d J 2.9), 118.8 (d J 2.9), 114.3 (d J 19.1); HRMS (FI) C₁₄H₁₁OF (M) requires 214.0794, found 214.0794.

### (E)-4-(4-Methoxystyryl)phenol 87a⁷⁶

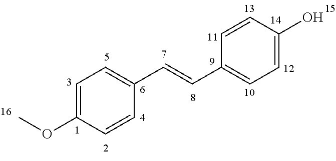 [SMILES: COc1ccc(/C=C/c2ccc(O)cc2)cc1]

Following general procedure 3, 4-iodophenol (219 mg, 0.995 mmol) and styrene 30a (200 mg, 1.49 mmol) gave the title compound as a brown solid (129 mg, 57%). The analytical data were in accordance with those reported in the lit.⁷⁶ mp 207-209° C.; Umax (cm⁻¹) 3415 (w, br), 3019 (w), 1605 (m), 1510 (m), 1241 (m), 832 (s); OH (400 MHz) 7.47 (2H, apparent ddd J 8.8, 3.0, 2.1, arom.), 7.41 (2H, apparent ddd J 8.7, 2.9, 2.0, arom.), 7.02 (1 H, d J 16.4, alkene proton), 6.98 (1 H, d J 16.4, alkene proton), 6.91 (2H, apparent ddd J 8.8, 3.0, 2.1, arom.), 6.84 (2H, apparent ddd J 8.7, 2.9, 2.0, arom.), 3.79 (3H, s, H16), OH proton H15 not observed; δ_{C} (100 MHz) 160.0, 157.9, 131.5, 130.4, 128.5, 128.2, 127.2, 126.2, 116.4, 114.9, 55.6 (C16); m/z (ESI⁻) 225 ([M−H]⁻).

### (E)-4-(3-Methoxystyryl)phenol 87b⁷⁶

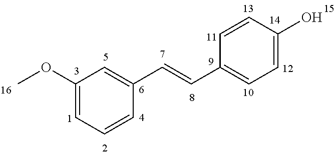 [SMILES: COc1cccc(/C=C/c2ccc(O)cc2)c1]

Following general procedure 3, 4-iodophenol (165 mg, 0.75 mmol) and styrene 30b (150 mg, 1.12 mmol) gave the title compound as a brown solid (153 mg, 90%). The analytical data were in accordance with those reported in the lit.⁷⁶ δ_{H} (400 MHz) 7.45 (2H, apparent ddd J 8.6, 2.9, 2.0, H12, H13), 7.25 (1H, m, arom.), 7.17 (1H, d J 16.4, H8), 7.12 (2H, m, arom.), 7.02 (1H, d J 16.4, H7), 6.86 (2H, d J8.6, H10, H11), 6.80 (1H, ddd J 8.1, 2.4, 0.8, arom.), 3.82 (3H, s, H16), OH proton H15 not observed; δ_{C} (100 MHz) 161.1, 158.5, 140.4, 130.4, 129.8, 129.7, 128.8 (C10, C11), 126.3, 119.6, 116.5 (C12, C13), 113.6, 112.1, 55.5 (C16); m/z (ESI⁻) 225 ([M−H]⁻).

### (E)-4-(2-Methoxystyryl)phenol 87c⁷⁶

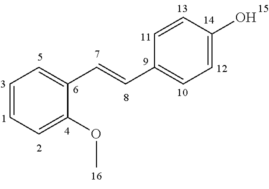 [SMILES: COc1ccccc1/C=C/c1ccc(O)cc1]

Following general procedure 3, 4-iodophenol (165 mg, 0.75 mmol) and styrene 30c (150 mg, 1.12 mmol) gave the title compound as a brown solid (90 mg, 53%). The analytical data were in accordance with those reported in the lit.⁷⁶ δ_{H} (400 MHz) 7.61 (1 H, dd J 7.8, 1.4, arom.), 7.43 (2H, apparent ddd J 8.6, 2.8, 2.0, H10, H11), 7.34 (1 H, d J 16.5, H8), 7.21 (1H, m, arom.), 7.13 (1H, d J 16.5, H7), 6.95 (2H, m, arom.), 6.86 (2H, apparent ddd J 8.6, 2.8, 2.0, H12, H13), 3.87 (3H, s, H16), OH proton H15 not observed; δ_{C} (100 MHz) 158.2, 157.7, 130.5, 129.6, 129.1, 128.7 (C10, C11), 127.5, 126.7, 121.5, 121.2, 116.5 (C12, C13), 111.9, 55.9 (C16); m/z (ESI⁻) 225 ([M−H]⁻).

### (E)-4-(4-Fluorostyryl)phenol 88a⁷⁶

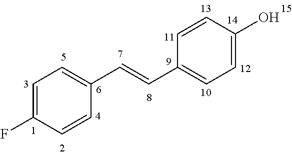 [SMILES: Oc1ccc(/C=C/c2ccc(F)cc2)cc1]

Following general procedure 3, 4-iodophenol (150 mg, 0.682 mmol) and styrene 95a (125 mg, 1.02 mmol) gave the title compound as a white solid (51 mg, 35%). The analytical data were in accordance with those reported in the lit.⁷⁶ δ_{H} (400 MHz) 8.51 (1H, broad s, H15), 7.58 (2H, apparent dddd J 8.8, 5.5, 3.0, 2.2, H4, H5), 7.45 (2H, apparent ddd J 8.6, 2.9, 2.0, H10, H11), 7.11 (3H, m, H2, H3, H8), 7.04 (1H, d J 16.5, H7), 6.85 (2H, apparent ddd J 8.6, 2.9, 2.0, H12, H13); O_{c} (100 MHz) 162.9 (d J 244.1, CI), 158.3 (C14), 135.5 (d J 2.8, C6), 130.0 (C9), 129.4 (d J 1.9, C7), 128.8 (010, 011), 128.8 (d J 7.4, C4, C5), 125.3 (C8), 116.5 (C12, C13), 116.3 (d J 22.2, C2, C3); m/z (ESI⁻) 213 ([M−H]⁻).

### (E)-4-(4-Nitrostyryl)phenol 94a⁸²

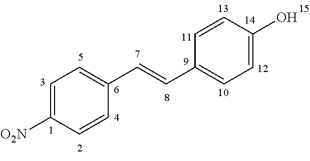 [SMILES: O=[N+]([O-])c1ccc(/C=C/c2ccc(O)cc2)cc1]

Following general procedure 6, styrene 97a (131 mg, 0.878 mmol) and 4-iodophenol (149 mg, 0.675 mmol) gave the title compound as an orange solid (101 mg, 62%). The analytical data were in accordance with those reported in the lit.⁸² δ_{H} (400 MHz) 8.72 (1H, br. s, H15), 8.18 (2H, apparent ddd J 8.9, 2.5, 1.9, H2, H3), 7.75 (2H, apparent ddd J 8.9, 2.5, 1.9, H4, H5), 7.52 (2H, apparent ddd J 8.7, 2.8, 2.0, H10, H11), 7.41 (1H, d J 16.4, H8), 7.16 (1H, d J 16.4, H7), 6.89 (2H, apparent ddd J 8.7, 2.8, 2.0, H12, H13); δ_{C} (100 MHz) 159.2 (C14), 147.2 (C1), 145.8 (C6), 134.2 (C8), 129.7 (C10, C₁₁), 129.2 (C9), 127.5 (C4, C5), 124.8 (C2, C3), 124.2 (C7), 116.7 (C12, C13); HRMS (FI) C₁₄H₁₁O₃N (M) requires 241.0739, found 241.0746.

### (E)-4-(3-Nitrostyryl)phenol 94b⁸³

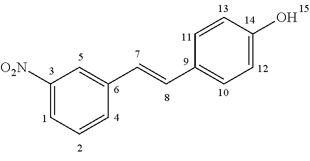 [SMILES: O=[N+]([O-])c1cccc(/C=C/c2ccc(O)cc2)c1]

Following general procedure 6, styrene 97b (145 mg, 0.972 mmol) and 4-iodophenol (165 mg, 0.748 mmol) gave the title compound as an orange solid (138 mg, 76%). The analytical data were in accordance with those reported in the lit.⁸³ δ_{H} (400 MHz) 8.64 (1H, br. s, H15), 8.37 (1H, apparent dd J 2.1, 1.9, H5), 8.06 (1H, ddd J 8.1, 2.1, 0.8, H1), 7.98 (1H, m, H4), 7.62 (1H, apparent dd J8.1, 8.0, H2), 7.53 (2H, apparent ddd J8.7, 2.8, 2.1, H10, H11), 7.40 (1H, d J 16.4, H8), 7.19 (1H, d J 16.4, H7), 6.88 (2H, apparent ddd J 8.7, 2.8, 2.1, H12, H13); δ_{C} (100 MHz) 158.9 (C14), 149.8 (C3), 141.0 (C6), 132.9 (C4), 132.5 (C8), 130.8 (C2), 129.4 (010, C11), 129.3 (C9), 124.1 (C7), 122.1 (C1), 121.2 (C5), 116.6 (C12, C13); HRMS (FI) C₁₄H₁₁O₃N (M) requires 241.0739, found 241.0737.

### (E)-4-(2-Nitrostyryl)phenol 94c⁸³

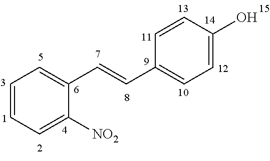 [SMILES: O=[N+]([O-])c1ccccc1/C=C/c1ccc(O)cc1]

Following general procedure 6, styrene 97c (146 mg, 0.979 mmol) and 4-iodophenol (166 mg, 0.753 mmol) gave the title compound as an orange solid (77 mg, 42%). The analytical data were in accordance with those reported in the lit.⁸³ δ_{H} (400 MHz) 8.67 (1H, br. s, H15), 7.93 (2H, m, arom.), 7.69 (1H, apparent dd J7.8, 7.6, arom.), 7.49 (3H, m, arom.), 7.38 (1 H, d J 16.2, H8), 7.24 (1 H, d J 16.2, H7), 6.89 (2H, apparent ddd J 8.7, 2.9, 2.0, H12, H13); δ_{C} (100 MHz) 159.1, 149.2, 134.6 (C7), 134.0, 133.6, 129.6 (C10, C11), 129.3, 128.6, 128.6, 125.4, 120.6 (C8), 116.6 (C12, C13); HRMS (FI) C₁₄H11O₃N (M) requires 241.0739, found 241.0742.

### 1-Fluoro-3-vinylbenzene 95a⁸⁴

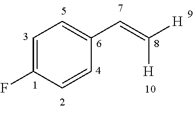

Following general procedure 4, 4-fluorobenzaldehyde (0.44 mL, 4.09 mmol) gave the title compound as a clear oil (101 mg, 20%). The analytical data were in accordance with those reported in the lit.⁸⁴ δ_{H} (400 MHz, CDCl₃) 7.38 (2H, apparent dddd J 8.8, 5.5, 3.1, 2.1, H4, H5), 7.03 (2H, apparent dddd J 8.8, 8.7, 3.1, 2.1, H2, H3), 6.69 (1H, dd J 17.6, 10.8, H7), 5.68 (1H, d J 17.6, H10), 5.23 (1H, d J 10.8, H9); δ_{C} (100 MHz, CDCl₃) 162.3 (d J 246.4, C1), 135.5 (C7), 133.6 (d J 4.0, C6), 127.6 (d J 8.0, C4, C5), 115.2 (d J 21.5, C2, C3), 113.3 (C8); HRMS (FI) C₈H₇F (M) requires 122.0532, found 122.0530.

### 1-Fluoro-3-vinylbenzene 95b⁸⁵

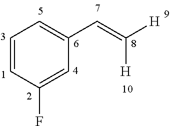

Following general procedure 4, 3-fluorobenzaldehyde (0.43 mL, 4.09 mmol) gave the title compound as a clear oil (180 mg, 36%). The analytical data were in accordance with those reported in the lit.⁸⁵ δ_{H} (400 MHz, CDCl₃) 7.18 (1H, m, arom.), 7.04 (2H, m, arom.), 6.86 (1H, apparent ddd J 8.5, 8.4, 2.3, arom.), 6.59 (1H, dd J 17.6, 10.8, H7), 5.67 (1H, d J 17.6, H10), 5.20 (1H, d J 10.8, H9); Oc (100 MHz, CDCI₃) 162.8 (d J 245.0, C2), 139.6 (d J 7.3), 135.5 (d J 2.9), 129.6 (d J 8.8), 121.8 (d J 2.2), 114.7, 114.2 (d J 21.3), 112.2 (d J22.0); HRMS (FI) C₈H₇F (M) requires 122.0532, found 122.0529.

### 1-Nitro-4-vinylbenzene 97a⁸⁷

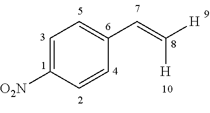 [SMILES: C=Cc1ccc([N+](=O)[O-])cc1]

Following general procedure 4, 4-nitrobenzaldehyde (506 mg, 3.35 mmol) gave the title compound as a clear oil (308 mg, 62%). The analytical data were in accordance with those reported in the lit.⁸⁷ δ_{H} (400 MHz) 8.21 (2H, d J8.5, H2, H3), 7.74 (2H, d J8.5, H4, H5), 6.90 (1 H, dd J 17 .7 , 11.0, H7), 6.08 (1 H, d J 17 .7 , H10), 5.52 (1 H, d J 11.0, H9); δ_{C} (100 MHz) 148.2 (C1), 144.9 (C6), 136.1 (C7), 128.0 (C2, C3), 124.7 (C4, C5), 119.2 (C8); HRMS (FI) C₈H₇NO₂ (M) requires 149.0477, found 149.0476.

### 1-Nitro-3-vinylbenzene 97b⁸⁸

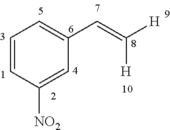 [SMILES: C=Cc1cccc([N+](=O)[O-])c1]

Following general procedure 4, 3-nitrobenzaldehyde (506 mg, 3.35 mmol) gave the title compound as a clear oil (178 mg, 56%). The analytical data were in accordance with those reported in the lit.⁸⁷ δ_{H} (400 MHz) 8.28 (1H, s, arom.), 8.12 (1H, m, arom.), 7.91 (1H, s, arom.), 7.64 (1H, m, arom.), 6.90 (1H, m, H7), 6.04 (1H, d J17.7, H10), 5.46 (1H, d J 11.0, H9); δ_{C} (100 MHz) 149.7 (C2), 140.3, 135.9 (C7), 133.0, 130.8, 123.2, 121.6, 117.6 (C8); HRMS (FI) C₈H₇NO₂ (M) requires 149.0477, found 149.0473.

### 1-Nitro-2-vinylbenzene 97c⁸⁹

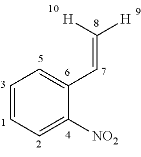 [SMILES: C=Cc1ccccc1[N+](=O)[O-]]

Following general procedure 4, 2-nitrobenzaldehyde (506 mg, 3.35 mmol) gave the title compound as a yellowish oil (390 mg, 78%). The analytical data were in accordance with those reported in the lit.⁸⁹ O_{H} (400 MHz) 7.94 (1 H, d J8.2, H2), 7.80 (1H, d J 7.9, H5), 7.70 (1H, m, H3), 7.55 (1H, m, H1), 7.10 (1H, dd J 17.4, 11.0, H7), 5.87 (1H, d J 17.4, H10), 5.50 (1H, d J 11.0, H9); OH (100 MHz) 149.2 (C4), 134.1 (C6), 133.4 (C3), 133.0 (C7), 129.7 (H1), 129.1 (C5), 125.1 (C2), 119.5 (C8); HRMS (FI) C₈H₇NO₂ (M) requires 149.0477, found 149.0476.

### Preparation and Characterisation of Compounds

### EXAMPLE 1

### Preparation of (E)-N,N-dimethyl-3-(4-styrylphenoxy)propan-1-amine 15a

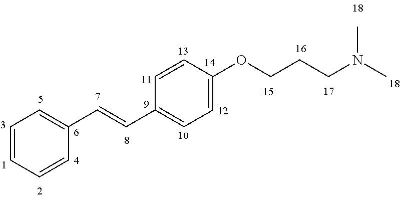 [SMILES: CN(C)CCCOc1ccc(/C=C/c2ccccc2)cc1]

n-BuLi (2.5M in hexanes, 0.57 mL, 1.42 mmol) was added to a stirred solution of diethyl benzylphosphonate (0.3 mL, 1.42 mmol) in THF (2 mL) at 0° C., and stirred for 30 min. A solution of the aldehyde 17a (0.24 mL, 1.21 mmol) in THF was then added dropwise to the reaction, which was then stirred for 16 h. The reaction mixture was quenched with NH₄Cl (sat. aq. sol. 20 mL) and extracted with EtOAc (3×30 mL). The organic phase was dried (Na₂SO₄), filtered and concentrated in vacuo to yield the crude product. Purification by recrystallisation in Pet Ether (40-60° C.) afforded the title compound as a white solid (22 mg, 7%). mp 75-77° C.; uₘₐₓ (cm⁻¹) 2940 (w), 2774 (w), 1687 (w), 1602 (m), 1510 (s), 1246 (s), 1178 (s), 1055 (s); δ_{H} (400 MHz, DMSO-d6) 7.56 (2H, m, arom.), 7.53 (2H, apparent ddd J 8.7, 2.9, 2.1, H10, H11) 7.36 (2H, m, arom.), 7.23 (2H, m, arom.), 7.08 (1H, d, J 16.3, H7), 6.94 (2H, apparent ddd J 8.7, 2.9, 2.1, H12, H13), 4.01 (2H, t J 6.5, H15), 2.44 (2H, t, J 7.0, H17), 2.21 (6H, s, H18), 1.87 (2H, m, H16); δ_{C} (100 MHz, DMSO-d6) 158.4 (C14), 137.4 (C6), 129.6 (C9), 128.7 (C2, C3), 128.1 (C8), 127.8 (C10, C11), 127.2 (C1), 126.2 (C4, C5), 126.1 (C7), 114.7 (C12, C13), 65.7 (C15), 55.5 (C17), 44.9 (C18), 26.6 (C16); m/z (ESI⁺) 282 ([M+H]⁺); HRMS (ESI⁺) C₁₉H₂₄NO ([M+H]⁺) requires 282.1852, found 282.1855.

### EXAMPLE 2

### Preparation of (E)-1-((4-methylpentyl)oxy)-4-styrylbenzene 39

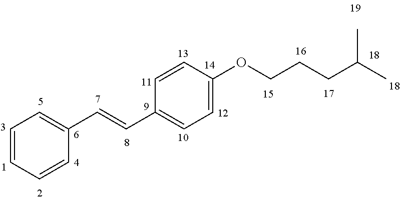 [SMILES: CC(C)CCCOc1ccc(/C=C/c2ccccc2)cc1]

Alcohol 32 (Example 16; 100 mg, 0.510 mmol, 1 eq) was dissolved in DMF (1 mL) in a microwave vial with K₂CO₃ (211 mg, 1.53 mmol, 3 eq) and stirred at 90° C. for 30 min. Tosylate 38 (144 mg, 0.561 mmol, 1.1 eq) was added, and the reaction was stirred at 90° C. for a further 16 h. After cooling, the reaction mixture was diluted with H₂O (10 mL) and extracted with EtOAc. The combined organics were dried (Na₂SO₄), filtered and concentrated in vacuo to yield the crude product. Purification by column chromatography (0.3%-4% EtOAc in Pet Ether) yielded the title compound as a white solid (114 mg, 80%). mp 97-98° C.; uₘₐₓ (cm⁻¹) 2955 (m), 1603 (m), 1510 (s), 1241 (s), 1175 (s), 967 (s); δ_{H} (400 MHz) 7.56 (2H, d J 7.5, H4, H5), 7.52 (2H, apparent ddd J 8.8, 2.9, 2.0, H10, H11), 7.35 (2H, m, H2, H3), 7.22 (2H, m, H1, H8), 7.09 (1H, d J 16.5, H7), 6.94 (2H, apparent ddd J 8.8, 2.9, 2.0, H12, H13), 3.99 (2H, t J 6.6, H15), 1.78 (2H, m, H16), 1.62 (1H, septet J 6.7, H18), 1.36 (2H, m, H17), 0.92 (6H, d J 6.7, H19); δ_{C} (125 MHz) 160.0 (C14), 138.8 (C6), 130.9 (C9), 129.6 (C2, C3), 129.2 (C8), 128.7 (C10, C11), 128.0 (C1), 127.1 (C4, C5), 127.1 (C7), 115.6 (C12, C13), 69.0 (C15), 36.0 (C17), 28.6 (C18), 28.0 (C16), 23.0 (C19); HRMS (FI) C₂₀H₂₄O (M) requires 280.1827, found 280.1829.

### EXAMPLE 3

### Preparation of (E)-1-(3-methoxypropoxy)-4-styrylbenzene 41

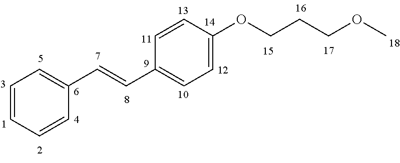 [SMILES: COCCCOc1ccc(/C=C/c2ccccc2)cc1]

Alcohol 40 (Example 17; 44 mg, 0.173 mmol, 1 eq.) in THF (0.5 mL) was added to a solution of NaH (140 mg, 3.46 mmol, 60% w/w in mineral oil, 20 eq.) in THF (0.5 mL), and left to stir for 1 h at RT. lodomethane (0.05 mL, 0.865 mmol, 5 eq.) was added, and the reaction was allowed to continue stirring for a further 16 h. The reaction mixture was then concentrated in vacuo to give the crude product; purification by flash chromatography yielded the title compound as a white solid (22 mg, 45%). mp 77.8-81.0° C.; uₘₐₓ (cm⁻¹) 2861 (w), 1604 (m), 1510 (s), 1123 (s); δ_{H} (400 MHz) 7.56 (2H, d J 7.9, H4, H5), 7.53 (2H, apparent ddd J 8.7, 2.9, 2.1, H10, H11), 7.35 (2H, apparent dd J 7.8, 7.5, H2, H3), 7.22 (2H, m, H1, H8), 7.09 (1 H, d J 16.5, H7), 6.94 (2H, apparent ddd J 8.7, 2.9, 2.1, H12, H13), 4.08 (2H, t J 6.3, H15), 3.53 (2H, t J 6.3, H17), 3.29 (3H, s, H18), 2.00 (2H, observed quintet, J 6.3, H16); δ_{C} (100 MHz) 159.9 (C14), 138.8, 131.0, 129.6, 129.1, 128.7, 128.0, 127.2, 127.1, 115.6, 69.68 (C15), 65.7 (C17), 58.7 (C18), 30.42 (C16); HRMS (FI) 018H2002 (M) requires 268.1463, found 268.1460.

### EXAMPLE 4

### Preparation of (E)-3-(4-(4-methoxystyryl)phenoxy)-N,N-dimethylpropan-1-amine 44a

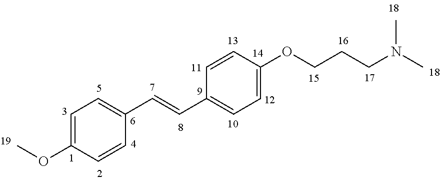

Following general procedure 1, stilbene 87a (50 mg, 0.221 mmol) and 3-dimethylamino-1-propylchloride hydrochloride (38 mg, 0.243 mmol) gave the title compound as a white solid (24 mg, 35%). mp 138-140° C.; uₘₐₓ (cm⁻¹) 2954 (w), 1605 (s), 1512 (s), 1247 (s), 1176 (s), 1030 (s); δ_{H} (400 MHz) 7.49 (4H, m, arom.), 7.04 (2H, s, H7, H8), 6.92 (4H, m, arom.), 4.05 (2H, t J 6.4, H15), 3.81 (3H, s, H19), 2.40 (2H, t J 7.0, H17), 2.18 (6H, s, H18), 1.91 (2H, m, H16); δ_{C} (100 MHz) 160.2, 159.7, 131.5, 131.4, 128.4, 128.4, 127.0, 126.9, 115.6, 115.0, 66.9 (C15), 56.9 (C17), 55.6 (C19), 45.8 (C18), 28.4 (C16); m/z (ESI⁺) 312 ([M+H]⁺); HRMS (ESI⁺) C₂₀H₂₆NO₂ ([M+H]⁺) requires 312.1958, found 312.1965.

### EXAMPLE 5

### Preparation of (E)-3-(4-(3-methoxystyryl)phenoxy)-N,N-dimethylpropan-1-amine 44b

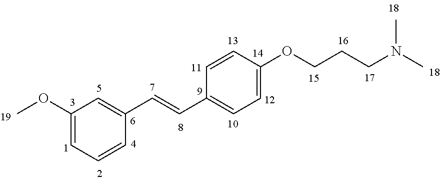

Following general procedure 1, alcohol 87b (100 mg, 0.441 mmol) and 3-dimethylamino-1-propylchloride hydrochloride (76 mg, 0.485 mmol) gave the title compound as a brown solid (71 mg, 52%). mp 28-32° C.; uₘₐₓ (cm⁻¹) 2944 (w), 2762 (w), 1598 (s), 1510 (s), 1254 (s), 1154 (s), 1042 (s); δ_{H} (400 MHz) 7.51 (2H, apparent ddd J 8.8, 2.8, 1.9, H10, H11), 7.26 (1H, m, H2), 7.20 (1H, d J 16.3, H7), 7.14 (2H, m, arom.), 7.06 (1H, d J 16.3, H8), 6.93 (2H, apparent ddd J 8.8, 2.8, 1.9, H12, H13), 6.82 (1H, m, arom.), 4.04 (2H, t J 6.4, H15), 3.82 (3H, s, H19), 2.39 (2H, t J 7.0, H17), 2.17 (6H, s, H18), 1.90 (2H, m, H16); δ_{C} (100 MHz) 161.1, 160.0, 140.2, 130.8, 130.5 (C8), 129.4 (C7), 128.7 (C10, C11), 127.1 (C2), 119.7, 115.6 (C12, C13), 113.8, 112.3, 66.9 (C15), 56.8 (C17), 55.5 (C19), 45.8 (C18), 28.3 (C16); m/z (ESL) 312 ([M+H]⁺); HRMS (ESL) C₂₀H₂₆NO₂ ([M+H]⁺) requires 312.1958, found 312.1957.

### EXAMPLE 6

### Preparation of (E)-3-(4-(2-methoxystyryl)phenoxy)-N,N-dimethylpropan-1-amine 44c

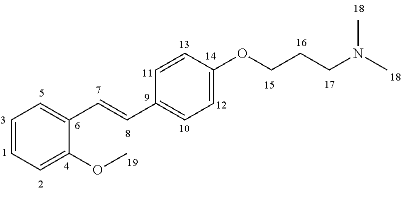 [SMILES: COc1ccccc1/C=C/c1ccc(OCCCN(C)C)cc1]

Following general procedure 1, alcohol 87c (71 mg, 0.313 mmol) and 3-dimethylamino-1-propylchloride hydrochloride (54 mg, 0.344 mmol) gave the title compound as a white solid (52 mg, 53%). mp 177.6-180.9° C.; uₘₐₓ (cm⁻¹) 2418 (w), 1604 (w), 1509 (m), 1240 (s), 1173 (s); δ_{H} (400 MHz) 7.63 (1H, dd J 7.7, 1.6, arom.), 7.51 (2H, apparent ddd J 8.8, 2.8, 2.0, H10, H11), 7.37 (1H, d J 16.6, H8), 7.23 (1H, m, arom.), 7.17 (1H, d J 16.6, H7), 6.97 (4H, m, arom.), 4.18 (2H, t J 6.1, H15), 3.89 (3H, s, H19), 3.27 (2H, m, H17), 2.80 (6H, s, H18), 2.36 (2H, m, H16); δ_{C} (125 MHz) 159.4, 157.9, 130.0, 129.3, 129.3, 128.6, 127.4, 126.9, 122.2, 121.6, 115.7 (C12, C13), 112.1, 66.3 (C15), 56.0 (C17), 55.4 (C19), 42.7 (C18), 25.3 (C16); m/z (ESI⁺) 312 ([M+H]⁺); HRMS (ESI⁺) C₂₀H₂₆NO₂ ([M+H]⁺) requires 312.1958, found 312.1959;

### EXAMPLE 7

### Preparation of (E)-3-(4-(4-fluorostyryl)phenoxy)-N,N-dimethylpropan-1-amine 46a

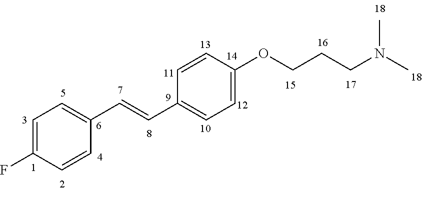 [SMILES: CN(C)CCCOc1ccc(/C=C/c2ccc(F)cc2)cc1]

Following general procedure 1, alcohol 88a (199 mg, 0.929 mmol) and 3-dimethylamino-1-propylchloride hydrochloride (161 mg, 1.02 mmol) gave the title compound as a white solid (232 mg, 83%); mp 128.8-129.9° C.; uₘₐₓ (cm⁻¹) 2951 (w), 2767 (w), 1510 (m), 1247 (m), 831 (s); δ_{H} (400 MHz, DMSO-d6) 7.60 (2H, m, arom.), 7.51 (2H, d J 8.6, H10, H11), 7.17 (3H, m, H8, arom.), 7.08 (1H, d J 16.5, H7), 6.93 (2H, d J 8.6, H12, H13), 4.00 (2H, t J 6.4, H15), 2.43 (2H, t J 7.2, H17), 2.20 (6H, s, H18), 1.87 (2H, m, H16); δ_{C} (125 MHz, DMSO-d6) 161.4 (d J 244.0, C1), 158.3 (C14), 134.0 (d J 2.8, C4, C5), 129.5, 128.0, 127.9, 127.7, 124.9, 115.5 (d J 21.3, C2, C3), 114.7 (C12, C13), 65.7 (C15), 55.5 (C17), 44.9 (C18), 26.6 (C16); m/z (ESL) 300 ([M+H]⁺); HRMS (ESI⁺) C₁₉H₂₃FNO ([M+H]⁺) requires 300.1758, found 300.1750;

### EXAMPLE 8

### Preparation of (E)-3-(4-(3-fluorostyryl)phenoxy)-N,N-dimethylpropan-1-amine 46b

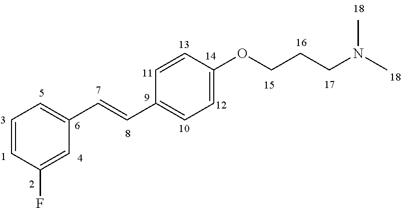 [SMILES: CN(C)CCCOc1ccc(/C=C/c2cccc(F)c2)cc1]

Pd(OAc)₂ (4 mg, 0.017 mmol, 0.05 eq.) and PPh₃ (13 mg, 0.051 mmol, 0.15 eq.) were added to a sealed microwave vial in DMF (1 mL), and the resulting solution was flushed with nitrogen. The resulting orange solution was then heated with stirring at 110° C. for 10 min. A solution of styrene 95b (55 mg, 0.434 mmol, 1.3 eq.), 4-iodophenol (73 mg, 0.334 mmol, 1 eq.) and K200₃ (185 mg, 1.34 mmol, 4 eq.) in DMF (0.5 mL) was then added, and the reaction mixture heated at 110° C. for a further 2 h. The reaction mixture was allowed to cool, then extracted with EtOAc. The combined organic layers were washed with brine, dried (Na₂SO₄), filtered and reduced in vacuo to give a solid (49 mg). This was redissolved in DMF (1 mL), treated with K₂CO₃ (94 mg, 0.681 mmol, 3 eq.) and heated at 90° C. for 30 min before addition of a solution of 3-dimethylamino-1-propylchloride hydrochloride (40 mg, 0.250 mmol, 1.1 eq.) neutralised previously with K₂CO₃ in DMF (0.5 mL). The microwave vial was sealed and heated at 90° C. for 16 h. After cooling, the reaction mixture was quenched with H₂O (10 mL), extracted with EtOAc (3×15 mL), the organic layer washed with brine, dried (Na₂SO₄) and concentrated in vacuo to yield the crude product. Purification by column chromatography (16% MeOH in CH₂Cl₂) afforded the desired product as a white solid (68 mg, 68%). mp 56.2-58.6° C.; uₘₐₓ (cm⁻¹) 2813 (w), 1600 (s), 1510 (s), 1253 (s), 1177 (s); OH (400 MHz) 7.55 (2H, apparent ddd J 8.7, 2.9, 2.0, H10, H11), 7.36 (3H, m, arom.), 7.27 (1H, d J 16.5, H8), 7.11 (1H, d J 16.5, H7), 6.98 (3H, m, arom.), 4.08 (2H, t J 6.4, H15), 2.52 (2H, t J 7.2, H17), 2.25 (6H, s, H18), 1.98 (2H, m, H16); δ_{C} (100 MHz) 164.2 (d J 243.0, C2), 160.3 (C14), 141.6 (d J 7.3), 131.3 (d J 8.8), 130.8, 130.6, 129.0 (C10, C11), 125.9 (d J 2.9), 123.4 (d J 2.9), 115.6 (C12, C13), 114.5 (d J 21.3), 113.1 (d, J 22.0), 66.8 (C15), 56.7 (C17), 45.4 (C18), 28.9 (C16); m/z (ESL) 300 ([M+H]⁺); HRMS (ESL⁺) C₁₉H₂₃NOF ([M+H]⁺) requires 300.1758, found 300.1754.

### EXAMPLE 9

### Preparation of (E)-N,N-dimethyl-3-(4-(4-nitrostyryl)phenoxy)propan-1-amine 47a

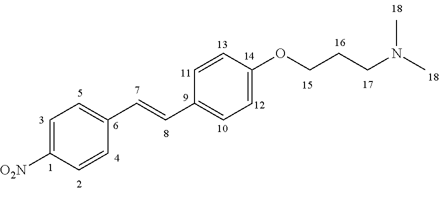 [SMILES: CN(C)CCCOc1ccc(/C=C/c2ccc([N+](=O)[O-])cc2)cc1]

Following general procedure 1, alcohol 94a (92 mg, 0.381 mmol) and 3-dimethylamino-1-propylchloride hydrochloride (66 mg, 0.419 mmol) gave the title compound as an orange solid (93 mg, 75%). mp 85.2-85.9° C.; uₘₐₓ (cm⁻¹) 2937 (w), 1589 (s, NO₂ asymmetric stretch), 1502 (s), 1332 (s, NO₂ symmetric stretch), 1174 (s); δ_{H} (400 MHz) 8.21 (2H, d J 8.7, H2, H3), 7.81 (2H, d J 8.7, H4, H5), 7.61 (2H, d J 8.6, H10, H11), 7.46 (1H, d J 16.4, H8), 7.24 (1H, d J 16.4, H7), 6.97 (2H, apparent ddd J 8.6, 3.1, 2.3, H12, H13), 4.08 (2H, t J 6.4, H15), 2.46 (2H, t J 7.1, H17), 2.22 (6H, s, H18), 1.94 (2H, m, H16); δ_{C} (100 MHz) 160.8 (C14), 147.3 (01), 145.7 (C6), 134.0 (C8), 130.1 (C9), 129.5 (C10, C11), 127.7 (C4, C5), 124.9, 124.9, 115.7 (C12, C13), 66.9 (C15), 56.7 (C17), 45.6 (C18), 28.1 (C16); m/z (ESL) 327 ([M+H]⁺); HRMS (ESI⁺) C₁₉H₂₃N₂O₃ ([M+H]⁺) requires 327.17032, found 327.16962.

### EXAMPLE 10

### Preparation of (E)-N,N-dimethyl-3-(4-(3-nitrostyryl)phenoxy)propan-1-amine 47b

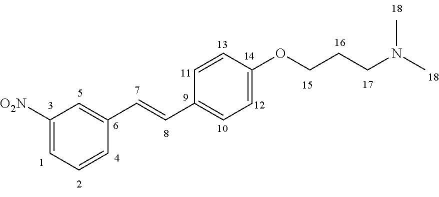 [SMILES: CN(C)CCCOc1ccc(/C=C/c2cccc([N+](=O)[O-])c2)cc1]

Following general procedure 1, alcohol 94b (129 mg, 0.535 mmol) and 3-dimethylamino-1-propylchloride hydrochloride (93 mg, 0.589 mmol) gave the title compound as a viscous orange oil (114 mg, 65%). uₘₐₓ (cm⁻¹) 2950 (w), 2765 (w), 1739 (m), 1600 (m), 1525 (s, NO₂ asymmetric stretch), 1353 (s, NO₂ symmetric stretch), 1245 (s); δ_{H} (400 MHz) 8.36 (1H, m, H5), 8.06 (1H, ddd J 8.2, 2.2, 0.8, H1), 7.97 (1H d J7.9, H4), 7.60 (3H, H2, H10, H11), 7.40 (1H, d J 16.4, H8), 7.21 (1H, d J 16.4, H7), 6.95 (2H, apparent ddd J8.8, 2.9, 2.0, H12, H13), 4.06 (2H, t J6.4, H15), 2.41 (2H, t J7.0, H17), 2.18 (6H, s, H18), 1.91 (2H, m, H16); δ_{C} (100 MHz) 160.5 (C14), 149.8 (C3), 140.8 (C6), 132.9 (C4), 132.2 (C8), 130.8 (C2), 130.1 (C9), 129.2 (010, C11), 124.7 (C7), 122.2 (C1), 121.3 (C5), 115.6 (C12, C13), 66.9 (C15), 56.8 (C17), 45.8 (C18), 28.3 (C16); m/z (ESI⁺) 327 ([M+H]⁺); HRMS (ESI⁺) C₁₉H₂₃N₂O₃ ([M+H]⁺) requires 327.17032, found 327.16960.

### EXAMPLE 11

### Preparation of (E)-N,N-dimethyl-3-(4-(2-nitrostyryl)phenoxy)propan-1-amine 47c

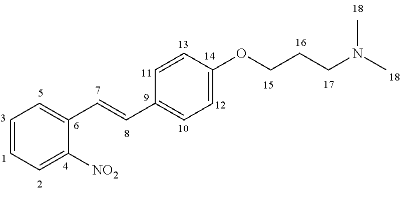 [SMILES: CN(C)CCCOc1ccc(/C=C/c2ccccc2[N+](=O)[O-])cc1]

Following general procedure 1, alcohol 94c (81 mg) and 3-dimethylamino-1-propylchloride hydrochloride (58 mg) gave the title compound as an orange solid (48 mg, 44%). mp 51.9-57.1° C.; uₘₐₓ (cm⁻¹) 2942 (w), 1599 (m, NO₂ asymmetric stretch), 1512 (s), 1342 (s, NO₂ symmetric stretch), 1250 (s); δ_{H} (400 MHz) 7.94 (2H, m, arom.), 7.69 (1H, apparent dd J 8.0, 7.3, arom.), 7.56 (2H, apparent ddd J 8.8, 2.9, 2.0, H10, H11), 7.49 (1 H, m, arom.), 7.41 (1 H, d J 16.2, H8), 7.27 (1 H, d J 16.2, H7), 6.97 (2H, apparent ddd J 8.8, 2.9, 2.0, H12, H13), 4.08 (2H, t J 6.4, H15), 2.51 (2H, t J7.1, H17), 2.25 (6H, s, H18), 1.96 (2H, m, H16); δ_{C} (100 MHz) 160.7 (C14), 149.2 (C4), 134.3 (C7), 134.0, 133.5, 130.2, 129.4 (C10, C11), 128.8, 128.6, 125.4, 121.3 (C8), 115.7 (C12, C13), 66.9 (C15), 56.6 (C17), 45.4 (C18), 27.9 (C16); m/z (ESL) 327 ([M+H]⁺); HRMS (ESL) C₁₉H₂₃N₂O₃ ([M+H]⁺) requires 327.17032, found 327.16966.

### EXAMPLE 12

### Preparation of (E)-3-(2-fluoro-4-styrylphenoxy)-N,N-dimethylpropan-1-amine 62

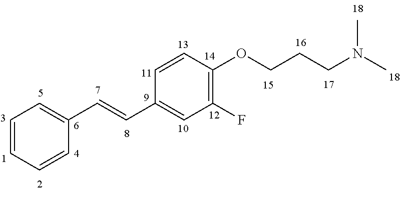 [SMILES: CN(C)CCCOc1ccc(/C=C/c2ccccc2)cc1F]

Following general procedure 1, alcohol 61 (53 mg, 0.247 mmol) and 3-dimethylamino-1-propylchloride hydrochloride (43 mg, 0.272 mmol) gave the title compound as a cream solid (31 mg, 42%). mp 68.2-72.6° C.; uₘₐₓ (cm⁻¹) 2940 (w), 2816 (m), 2766 (m), 1513 (s), 1273 (s), 1017 (s); OH (400 MHz) 7.57 (2H, d J 7.9, arom.) 7.44 (1H, dd J 12.9, 2.1, H10), 7.36 (2H, apparent dd J 7.9, 7.5, arom.), 7.31 (1H, d J 8.5, arom.), 7.23 (1H, m, arom.), 7.15 (3H, m, arom.), 4.14 (2H, t J 6.4, H15), 2.41 (2H, t J 6.9, H17), 2.17 (6H, s, H18), 1.93 (2H, m, H16); δ_{C} (125 MHz) 153.6 (d J 244.0, C12), 147.7 (d J 11.1, C14), 138.4, 131.9 (d J 6.5), 129.6, 128.7, 128.4, 128.2 (d J2.8), 127.3, 124.2 (d, J 2.8), 115.8, 114.1 (d J 19.4), 68.2 (C15), 56.7 (C17), 45.8 (C18), 28.3 (C16); m/z (ESI⁺) 300 ([M+H]⁺); HRMS (ESI⁺) C₁₉H₂₃NOF ([M₊H]⁺) requires 300.1758, found 300.1753.

### EXAMPLE 13

### Preparation of (E)-N-methyl-3-(4-styrylphenoxy)propan-1-amine 69

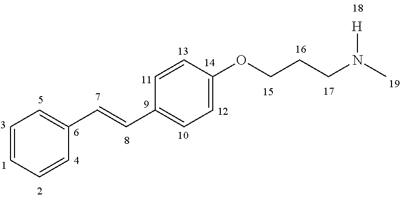 [SMILES: CNCCCOc1ccc(/C=C/c2ccccc2)cc1]

Stilbene 15a (Example 1; 40 mg, 0.142 mmol, 1 eq) was dissolved in CH2012(0.7 mL) to give a 0.2 M solution, and was added to 1-chloroethyl chloroformate (0.05 mL, 0.426 mmol, 3 eq) in a sealed microwave vial, to be heated to reflux for 16 h. After cooling, the reaction mixture was concentrated in vacuo. The residue was then redissolved in MeOH (1.5 mL), transferred so a sealed microwave vial, and refluxed for a further 2 h. The reaction mixture was left to cool, and concentrated in vacuo to yield the crude product. Purification by column chromatography (18% MeOH, 0.5% TEA in CH₂Cl₂) afforded the title compound as a cream solid (20 mg, 52%). mp 84.2-87.7° C.; uₘₐₓ (cm⁻¹) 2925 (w), 1623 (m), 1510 (s), 1384 (s), 1305 (s); OH (400 MHz, CD₃OD) 7.48 (4H, m, H4, H5, H10, H11), 7.32 (2H, apparent dd J 7.8, 7.5, H2, H3), 7.21 (1H, m, H1), 7.10 (1H, d J 16.5, H8), 7.00 (1H, d J 16.5, H7), 6.91 (2H, apparent ddd J 8.8, 2.9, 1.9, H12, H13), 4.04 (2H, t J 6.2, H15), 2.78 (2H, t J 7.2, H17), 2.42 (3H, s, H19), 1.99 (2H, m, H16), 1.27 (1H, broad s, H18); δ_{C} (100 MHz, CD₃OD) 160.2 (C14), 131.8, 129.8, 129.8, 129.4, 128.9, 128.3, 127.7, 127.4, 115.9 (C12, C13), 67.3 (C15), 49.7 (C17), 36.0 (C19), 29.9 (C16); m/z (ESL) 268 ([M+H]⁺); HRMS (ESL) Cl₈H₂₂NO ([M+H]⁺) requires 268.1696, found 268.1693

### EXAMPLE 14

### Preparation of (E)-4-styrylphenol 32⁵⁶

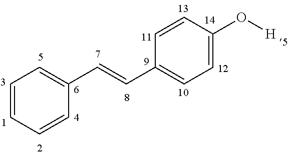 [SMILES: Oc1ccc(/C=C/c2ccccc2)cc1]

Following general procedure 2, 4-iodophenol (561 mg, 2.55 mmol) and styrene (0.35 mL, 3.06 mmol) gave the title compound as a white solid (122 mg, 24%). The analytical data were in accordance with those reported in the lit.⁵⁶ mp 180-182° C.; Umax (cm⁻¹) 3409 (w, br), 1591 (w), 1508 (w), 1450 (m), 1369 (m), 1245 (m, br), 960 (s); OH (400 MHz) 8.49 (1H, s, H15), 7.54 (2H, d J 8.0, H4, H5), 7.46 (2H, apparent ddd J 8.7, 2.9, 2.1, H10, H11), 7.34 (2H, m, H2, H3), 7.20 (2H, m, H1, H8), 7.05 (1H, d, J16.5, H7), 6.86 (2H, apparent ddd J 8.7, 2.9, 2.1, H12, H13); δ_{C} (100 MHz) 158.3 (C14), 138.9, 130.0, 129.5, 129.4, 128.8, 127.9, 127.0, 126.5, 116.5 (C12, C13); m/z (ESI⁻) 195 ([M−H]⁻).

### EXAMPLE 15

### Preparation of (E)-3-(4-styrylphenoxy)propan-1-ol 40⁵⁷

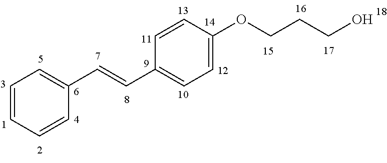 [SMILES: OCCCOc1ccc(/C=C/c2ccccc2)cc1]

Following general procedure 1, alcohol 32 (example 16; 100 mg, 0.51 mmol) and 3-chloro-1-propanol (0.05 mL, 0.56 mmol) gave the title compound as a white fluffy solid (92 mg, 71%). The analytical data were in accordance with those reported in the lit.⁵⁷ mp 166-167° C.; uₘₐₓ (cm⁻¹) 3275 (w. br. OH stretch), 1601 (m), 1507 (s), 1235 (s), 1043 (s); δ_{H} (400 MHz) 7.56 (2H, d J 7.7, H4, H5), 7.54 (2H, apparent ddd J 8.7, 2.9, 2.0, H10, H11), 7.35 (2H, m, H2, H3), 7.21 (2H, m, H1, H8), 7.09 (1H, d J 16.4, H7), 6.95 (2H, apparent ddd J 8.7, 2.9, 2.0, H12, H13), 4.13 (2H, t J 6.3, H15), 3.74 (2H, m, H17), 3.69 (1H, t J 5.2, H18), 1.97 (2H, m, H16); δ_{c} (100 MHz) 160.0 (C14), 138.8, 131.0, 129.6, 129.2, 128.7, 128.0, 127.1, 127.1, 115.6 (C12, C13), 65.7 (C15), 59.1 (C17), 33.4 (C16); m/z (ESL) 254 ([M−H]⁻); HRMS (FI ) C₁₇H₁₈O₂ (M) requires 254.1307, found 254.1317.

### EXAMPLE 16

### Preparation of (E)-1-methoxy-4-styrylbenzene 65⁵⁸

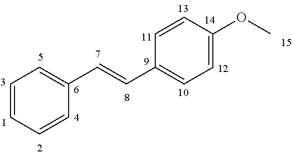 [SMILES: COc1ccc(/C=C/c2ccccc2)cc1]

Following general procedure 3, 4-iodoanisole (557 mg, 2.38 mmol) and styrene (0.33 mL, 2.85 mmol) gave the title compound as a white solid (236 mg, 47%). The analytical data were in accordance with those reported in the lit.⁵⁸ mp 129-132° C.; uₘ(cm⁻¹) 3003 (w), 1600 (m), 1508 (s), 1244 (s), 1177 (s); δ_{H} (400 MHz) 7.55 (4H, m, H4, H5, H10, H11), 7.35 (2H, m, H2, H3), 7.22 (2H, m, H1, H8), 7.10 (1H, d J 16.5, H7), 6.94 (2H, apparent ddd J 8.8, 3.0, 2.1, H12, H13), 3.81 (3H, s, H15); δ_{C} (100 MHz) 160.5 (C14), 138.8, 131.1, 129.6, 129.1, 128.7, 128.1, 127.2, 127.1, 115.0 (C12, C13), 55.7 (C15); HRMS (FI) C₁₅H₁₄O (M) requires 210.1045, found 210.1052.

### Biological Evaluation

### Luciferase Assay

Chinese Hamster Ovary (CHO) cells cultivated in 2×75 cm³ conical flasks had their media removed and washed in 5 mL of PBS before 2 mL of Trypsin was added. The cells were swirled for a minute, before being placed in the 37° C. incubator for 5 min. The conical flasks were tapped to remove cells from the bottom, and aspirated out into 2×13 mL of Ham F-12 media containing 10% FBS and 1% L-Glutamine. The cells were then spun down at 1000 rpm for 5 min. The media was aspirated off, and the pellet from each of the two flasks was resuspended in 5 mL of media. The cells were counted, plated into a 24-well plate to give 1.5×10⁵ cells/well in 500 μL of media, and placed in the 37° C. incubator. 2 mL of the remaining cells was added to 38 mL of media, plated 20 mL each into 2×75 cm² conical flasks, and placed in the 37° C. incubator.

After 24 h, the media was aspirated out and replaced with 500 μL Lipoprotein Deficient Serum (LPDS) media. The plate was then returned to the incubator for a further 24 h.

Following this incubation, the compound to be tested was dissolved in DMSO to give a concentration of 1 mg/mL. An appropriate amount of this solution was then added to FPS media to give 5 mL at a concentration of 10 μM. Serial dilution then gave the other required concentrations, to give testing concentrations of 0.1 nM, 1 nM, 10 nM, 30 nM, 100 nM, 1 μM and 10 μM. A 1 μM compound 49 positive control was also prepared from a stock solution in DMSO, and 0.1% DMSO control was also prepared as a negative control. Each well was then treated with 500 μL of each solution, according to the plate map shown below, and the plates were placed in the incubator.

48 hours after treatment, the cell media was aspirated and cells were washed with 500 μL of PBS. 100 μL of luciferase lysis buffer (12.5 mL 1M TrisPO₄ pH 7.8, 5 mL 0.2M CDTA solution, 50 mL glycerol, 5 mL Triton-X 100, 427.5 mL H₂O) was added to each sample; samples were shaken for 20 min at 4° C. before being pipetted up and down, then the plate was spun at 4500 rpm for 5 min. 75 μL of lysate was pippetted into a 96-well plate. The plate was then run on a Dynex Technologies MLX 96 Well Plate Luminometer using revalation software. 1004 of luciferase assay buffer solution and 50 μL of Luciferin solution (200 μL Luciferin 10X, 1800 μL H₂O) was dispensed each time. Response was recorded in Relative Light Units (RLUs), normalised to standards from a bicinchoninic acid (BCA) assay.

### Luciferase Assay Buffer Solution

Prepared using 3 mL luciferase assay buffer (7.5 mL 1M MgSO₄, 7.5 mL 1M TrisPO₄ pH 7.8 buffer, 20 mL 0.1M EGTA pH 7.8, 465 mL H₂O), added to 60 μL of 100 mM ATP and 6 μL of 1 M DTT solution.

### Results

Using the luciferase assay, the compounds of the invention provided the following activities:

| Example No. | Compound No. | EC50 (nM) | Eₘₐₓ |
|---|---|---|---|
| 1 | 15a | 39 | 5 |
| 2 | 39 | 1000 | 8 |
| 3 | 41 | 1500 | 44 |
| 4 | 44a | 260 | 30 |
| 5 | 44b | 2500 | 9 |
| 6 | 44c | 310 | 8 |
| 7 | 46a | 2000 | 24 |
| 8 | 46b | 1500 | 9 |
| 9 | 47a | 3000 | 39 |
| 10 | 47b | N/A | N/A |
| 11 | 47c | 3000 | 5 |
| 12 | 62 | 3000 | 15 |
| 13 | 69 | 320 | 8 |
| 14 | 32 | 3000 | 34 |
| 15 | 40 | 35 | 32 |
| 16 | 65 | 50 | 6 |

In general, the compounds of the present invention possess EC₅₀ values of 10 μM or less in the above-mentioned luciferase assay.

Preferred compounds of the invention possess EC₅₀ values of 3 μM or less in the above-mentioned luciferase assay.

The most preferred compounds possess EC₅₀ values of 500 nM or less in the above-mentioned luciferase assay.

In general, the compounds of the present invention possess E_{Max} values of 5 or greater in the above-mentioned luciferase assay.

Preferred compounds of the invention possess E_{Max} values of 8 or greater in the above-mentioned luciferase assay.

The most preferred compounds possess E_{Max} values of 20 or greater in the above-mentioned luciferase assay.

### Squalene Synthase Inhibition

Squalene Synthase activity assays were performed in 1 mL reactions containing 50 mM phosphate buffer (pH 7.4, 10 mM MgCl₂), 12 μg human liver microsomes, [1-³H]FPP (0.045 Ci/mmol) and 0.5 mM NADPH. Enzyme and inhibitors were incubated for 10 minutes at 37° C. Substrate was then added and reaction was incubated for 10 min at 37° C. Reaction was stopped by adding 1 mL of 15% KOH in ethanol and incubated at 65° C. for 30 minutes. 5 mL of petroleum ether was added and reaction was shaken for 10 minutes at room temperature. The lower aqueous phase was frozen and the upper organic phase transferred to fresh glass tubes containing 2 mL distilled water. 1.5 mL of the organic phase was transferred to a scintillation vial containing liquid scintillation fluid, and radioactivity was quantitated using a Beckman liquid scintillation counter. Data was analyzed using Prism Graphpad software.

As a representative example, compound 40 (Example 15; JM-086) was tested for squalene synthase inhibitory properties alongside compound 15a (Example 1), and the known squalene synthase inhibitor, YM-53601, as a positive control.

### Results

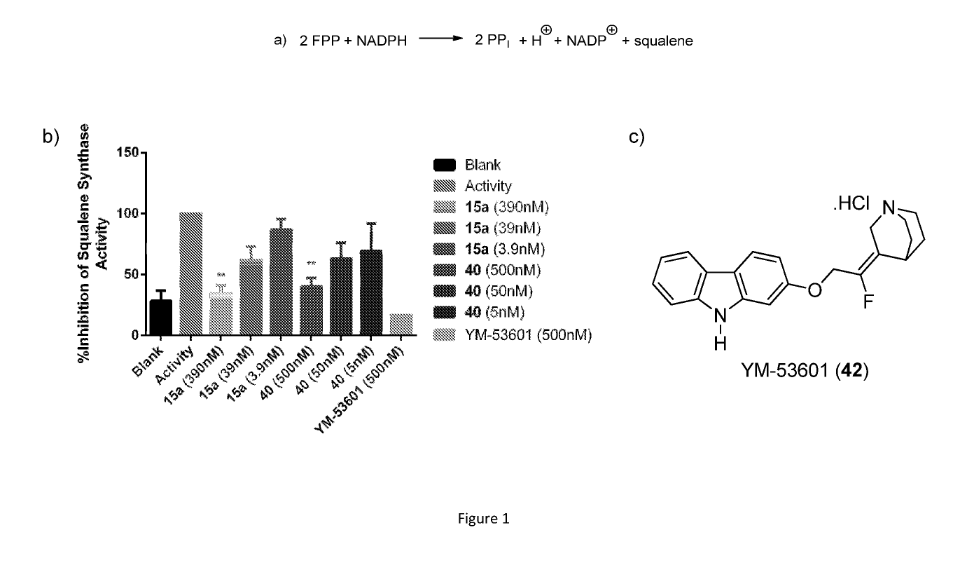

The results are shown in FIG. 1.

FIG. 1a) shows the reaction scheme of farnesyl pyrophosphate (FPP) to squalene; FIG. 1b) shows the results of the squalene synthase assay; Blank: contains [³H] FPP and NADPH. Activity: contains microsomes, [³H] FPP and NADPH; FIG. 1c) shows the chemical structure of YM-53601, a known squalene synthase inhibitor used as a positive control.

It can be seen from these results that compound 15a (Example 1) and compound 40 (Example 15; JM-086) both show dose-dependent activity, demonstrating that they are indeed squalene synthase inhibitors.

### In Silico Modeling

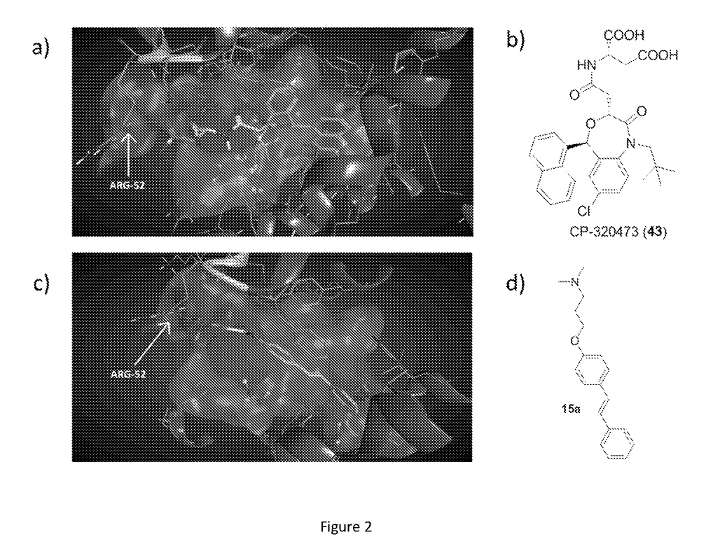

In silico modeling using MOE⁴⁶ was performed against a protein crystal structure of human squalene synthase (PDB: 1EZF),⁴⁷ which had been co-crystallised with the ligand CP-320473 (FIG. 2a). 15a (Example 1) was docked in its neutral state.

FIG. 2 shows: a) CP-320473 co-crystallised with human squalene synthase; b) the structure of CP-320473; c) in silico docking of 15a (Example 1) into the active site of human squalene synthase; d) the structure of 15a (Example 1).

It can be seen from FIG. 2 that 15a (Example 1) is predicted to bind in a similar manner as that determined for the co-ligand, with the hydrophobic aryl group sitting deep in the pocket while the chain bearing hydrogen bond acceptors protrudes out to interact with Arg-52. The close proximity of the compound to this basic residue would suggest replacement of amine group with an acidic one could improve activity as an ionic interaction should stabilise the binding inside the active site, and this will be tested in the future.

### Combination Therapy Experiments

CHO pLDLR-Luciferase Cells Used for Luciferase Compound Screen

As described above, CHO WT cells transfected with pLDLR-Luc (CHO-pLDLR-Luc) were grown in HamsF-12(Sigma N4888) medium supplemented with 10% fetal bovine serum (FBS), 1% penicillin/streptomycin, 1% L-glutamine, in a 5% CO₂ incubator at 37° C. CHO-pLDLR-Luc were seeded in 24-well plates at 1.5×10⁵ per well. After 24h cells were changed to HamsF-12(Sigma N4888) supplemented with 10% lipoprotein deficient serum (LDPS), 1% penicillin/streptomycin, 1% L-glutamine. Compounds and simvastatin (Sigma S6196) dissolved in DMSO and Cholesterol(Sigma C8667) and 25-Hydroxycholesterol(Sigma H1015) dissolved in ethanol were added 24 h after serum starvation.

Simvastatin and compound 40 (Example 15; JM-086) were added together. The compounds were added at the exact same time, with the EC₅₀ of either compound 40 (JM-086) or Simvastatin added into the media containing increasing doses of either compound 40 (JM-086) or Simvastatin then being added to the CHO pLDLR-Luciferase cells.

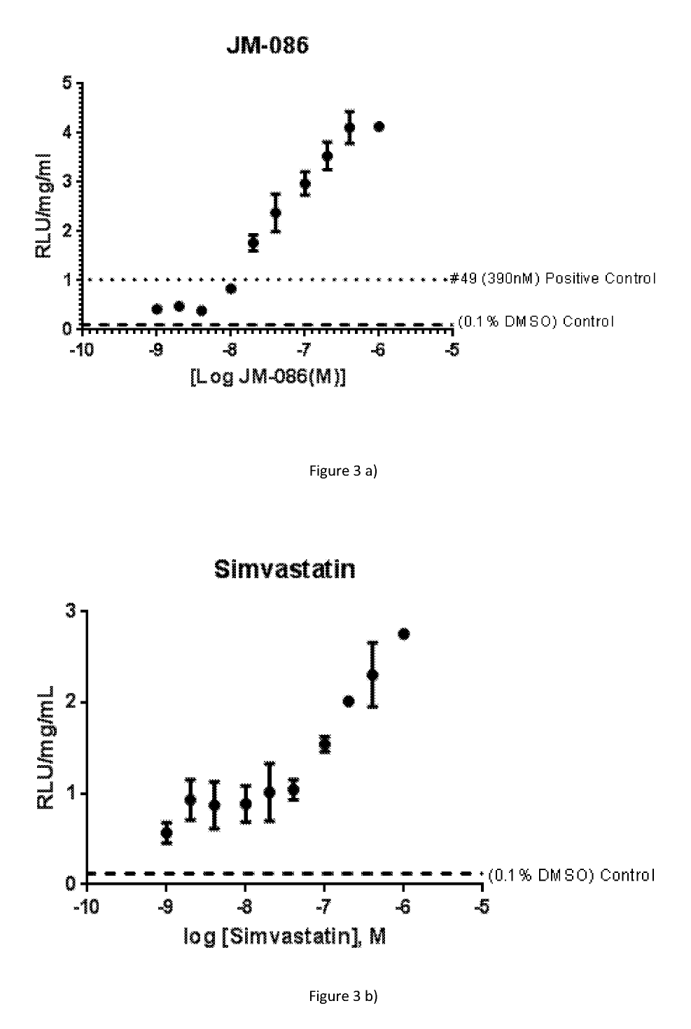

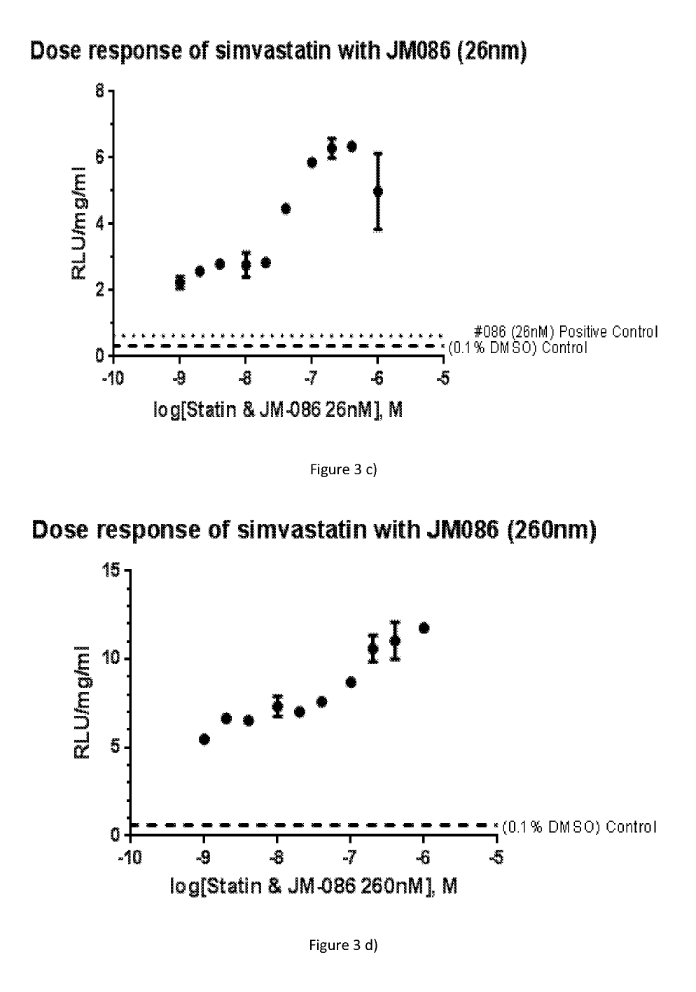

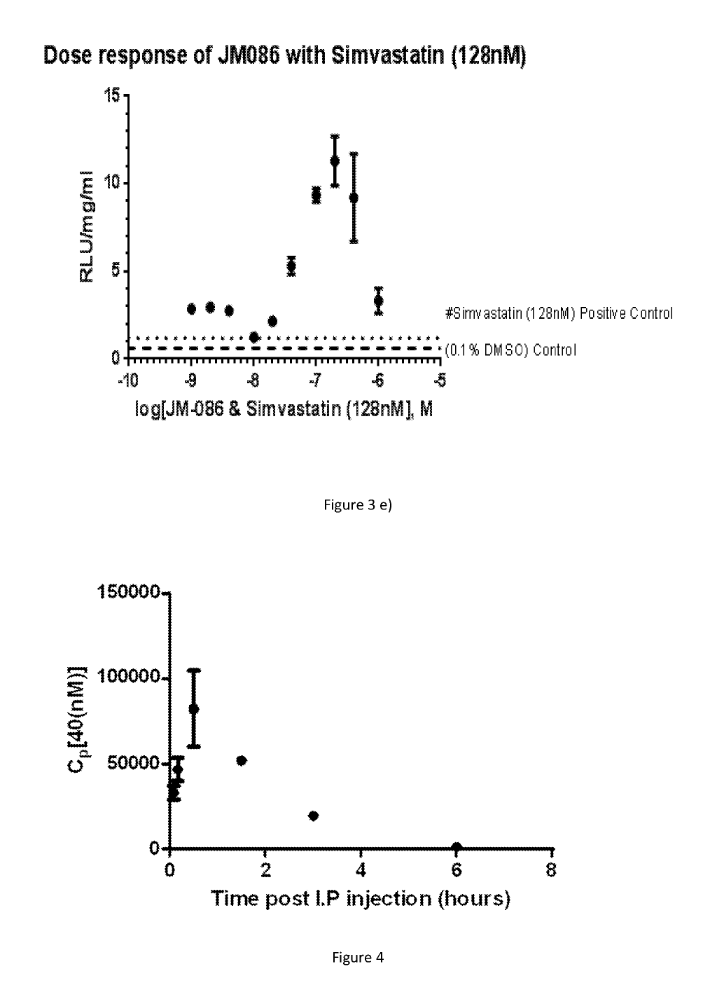

The results are shown in FIG. 3a) to e).

FIG. 3a) shows a dose-dependent increase in levels of luciferase when increasing concentrations of compound 40 (Example 15; JM-086; EC50 (n=2 ±SEM)=31.5 nM).

FIG. 3b) shows a dose-dependent increase in levels of luciferase when increasing concentrations of Simvastatin. The EC50 (n=1) was calculated at (EC50 (n=1±SEM)=128 nM) are added.

FIG. 3c) shows a dose-dependent increase in levels of luciferase when increasing concentrations of simvastatin with compound 40 (JM-086; EC50 (26 nM)) are added. The amount of luciferase produced is greatly increased when compounds are combined, rather than when on there own. Noted with the Eₘₐₓ being 2 fold greater.

FIG. 3d) shows a dose-dependent increase in levels of luciferase when increasing concentrations of Simvastatin with compound 40 (JM-086; high dose (260 nM)) are added. The amount of luciferase produced is greatly increased when compounds are combined, rather than when on there own. Noted with the Eₘₐₓ being 4 fold greater.

FIG. 3e) shows a dose-dependent increase in levels of luciferase when increasing concentrations of compound 40 (Example 15; JM-086) are added with Simvastatin (EC50(128 nM)) are added. The amount of luciferase produced is greatly increased when compounds are combined, rather than when on there own. Noted with the Emax being 3 fold greater.

### Quantification of Example 15 (Compound 40) in Mouse Plasma

Compound 40 (Example 15; JM-086) was quantified in mouse plasma using HPLC with absorbance detection. Terminal blood samples were taken from CD-1 mice that had been dosed with compound 40 via the intraperitoneal route for various time points. The samples were collected in Microvette CB 300 LH (Starsedt) capillary tubes and centrifuged at for 15 minutes at 3000×g and 4° C. before the plasma fraction was stored at −80° C. Upon analysis plasma samples were diluted 1:8 in acetonitrile and spun at 15 minutes at 13,000rpm and 4° C. 200 μL of the supernatant from each sample was then injected onto an isocratic HPLC system and quantified using absorbance detection. HPLC separation was performed using a reverse phase column (WaterS and Terra RP18 5 mm) and a linear gradient mobile phase starting at 94.9% H₂O 0.1% TFA 5% Acetonitrile changing to 4.9% H₂O 0.1% TFA 95% Acetonitrile over 10 minutes at a flow rate of 1.5 ml/min. Elution of compound 40 was measured by absorbance at 300 nm using an absorbance reader (Waters 2487 absorbance detector). Compound 40 elution was identified and quantified relative to known compound 40 concentrations spiked into mouse plasma and prepared as stated above and quantified using the peak area using Millennium32 software version 3.2 (Waters).

The results are shown in FIG. 4, which shows the plasma concentration of over time following the administration of compound 40 (Example 15; JM-086) by intraperitoneal (I.P.) injection at a dose of 40 mg/kg.

### REFERENCES

1. M. Prashad, F. G. Kathawala and T. Scallen, J. Med. Chem., 1993, 36, 1501-1504.
2. T. Ishihara, H. Kakuta, H. Moritani, T. Ugawa and I. Yanagisawa, Bioorg. Med. Chem., 2004, 12, 5899-5908.
3. C. K. Lim, Z. X. Yuan, J. H. Lamb, I. N. H. White, F. De Matteis and L. L. Smith, Carcinogenesis, 1994, 15, 589-593.
4. M. Ezzahti, E. J. G. Sijbrands, M. T. Mulder and J. E. R. van Lennep, Neth. J. Med., 2013, 71, 227-233.
5. G. K. Hovingh, M. H. Davidson, J. J. P. Kastelein and A. M. O'Connor, Eur. Heart J., 2013, 34, 962-971.
6. F. J. Raal, G. J. Pilcher, D. R. Illingworth, A. S. Pappu, E. A. Stein, P. Laskarzewski, Y. B. Mitchel and M. R. Melino, Atherosclerosis, 1997, 135, 249-256.
7. B. G. Nordestgaard, M. J. Chapman, S. E. Humphries, H. N. Ginsberg, L. Masana, O. S. Descamps, O. Wiklund, R. A. Hegele, F. J. Raal, J. C. Defesche, A. Wiegman, R. D. Santos, G. F. Watts, K. G. Parhofer, G. K. Hovingh, P. T. Kovanen, C. Boileau, M. Averna, J. Boren, E. Bruckert, A. L. Catapano, J. A. Kuivenhoven, P. Pajukanta, K. Ray, A. F. H. Stalenhoef, E. Stroes, M. R. Taskinen and A. Tybjaerg-Hansen, Eur. Heart J., 2013, 34, 3478-3490.
8. T. J. Smilde, S. van Wissen, H. Wollersheim, J. J. P. Kastelein and A. F. H. Stalenhoef, Neth. J. Med., 2001, 59, 184-195.
9. World Health Organisation, Global status report on noncommunicable diseases 2010, 2010.
10. A. C. M. Jansen, S. Van Wissen, J. C. Defesche and J. J. P. Kastelein, Curr. Opin. Lipidol., 2002, 13, 165-171.
11. National Institute for Health and Clinical Excellence, Clinical guidelines and evidence review for familial hypercholesterolaemia: the identification and management of adults and children with familial hypercholesterolaemia, www.nice.org.uk/CG71.
12. P. Goyal, L. I. Igel, K. LaScalea and W. B. Borden, Curr. Atheroscler. Rep., 2014, 16, 390/391-390/312.
13. V. W. Rodwell, M. J. Beach, K. M. Bischoff, D. A. Bochar, B. G. Darnay, J. A. Friesen, J. F. Gill, M. Hedl, T. Jordan-Starck, P. J. Kennelly, D. Y. Kim and Y. Wang, Methods Enzymol., 2000, 324, 259-280.
14. B. Yeganeh, E. Wiechec, S. R. Ande, P. Sharma, A. R. Moghadam, M. Post, D. H. Freed, M. Hashemi, S. Shojaei, A. A. Zeki and S. Ghavami, Pharmacol. Ther., 2014, 143, 87-110.
15. A. M. Gotto and J. E. Moon, Nat. Rev. Cardiol., 2013, 10, 560-570.
16. S. Goswami, A. S. Vidyarthi, B. Bhunia and T. Mandal, J. Biochem. Technol., 2012, 4, 581-587.
17. E. S. Istvan and J. Deisenhofer, Science (Washington, D.C., U. S.), 2001, 292, 1160-1164.
18. National Institute for Health and Clinical Excellence, Statins for the prevention of cardiovascular events in patients at increased risk of developing cardiovascular disease or those with established cardiovascular disease, http://www.nice.org.uk/TA094.
19. J. Versmissen, D. M. Oosterveer, M. Yazdanpanah, J. C. Defesche, D. C. G. Basart, A. H. Liem, J. Heeringa, J. C. Witteman, P. J. Lansberg, J. J. P. Kastelein and E. J. G. Sijbrands, Bmj, 2008, 337, a2423-a2423.
20. A. H. Pijlman, R. Huijgen, S. N. Verhagen, B. P. M. Imholz, A. H. Liem, J. J. P. Kastelein, E. J. Abbink, A. F. H. Stalenhoef and F. L. J. Visseren, Atherosclerosis, 2010, 209, 189-194.
21. C. P. Cannon, E. Braunwald, C. H. McCabe, D. J. Rader, J. L. Rouleau, R. Belder, S. V. Joyal, K. A. Hill, M. A. Pfeffer and A. M. Skene, New Engl. J. Med., 2004, 350, 1495-1504.
22. J. J. P. Kastelein, F. Akdim, E. S. G. Stroes, A. H. Zwinderman, M. L. Bots, A. F. H. Stalenhoef, F. L. J. Visseren, E. J. G. Sijbrands, M. D. Trip, E. A. Stein, D. Gaudet, R. Duivenvoorden, E. P. Veltri, A. D. Marais and E. de Groot, New Engl. J. Med., 2008, 358, 1431-1443.
23. C. T. T. C. Collaboration, The Lancet, 2010, 376, 1670-1681.
24. G. Fernandez, S. Spatz Erica, C. Jablecki and S. Phillips Paul, Cleve. Clin. J. Med., 2011, 78, 393-403.
25. A. Jackevicius Cynthia, M. Mamdani and V. Tu Jack, Jama, 2002, 288, 462-467.
26. R. K. Elsayed and J. D. Evans, Expert Opin. Emerging Drugs, 2008, 13, 309-322.
27. T. Nishimoto, Y. Amano, R. Tozawa, E. Ishikawa, Y. Imura, H. Yukimasa and Y. Sugiyama, Br. J. Pharmacol., 2003, 139, 911-918.
28. N. Griebenow, A. Buchmueller, P. Kolkhof, J. Schamberger and H. Bischoff, Bioorg. Med. Chem. Lett., 2011, 21, 3648-3653.
29. E. Ladopoulou, A. N. Matralis and A. P. Kourounakis, J. Med. Chem., 2013, 56, 3330-3338.
30. E. A. Stein, H. Bays, D. O'Brien, J. Pedicano, E. Piper and A. Spezzi, Circulation, 2011, 123, 1974-1985.
31. V. S. Eligar, L. N. R. Bondugulapati and A. Rees, Curr. Opin. Lipidol., 2014, 25, 237-238.
32. K. S. Jain, M. K. Kathiravan, R. S. Somani and C. J. Shishoo, Bioorg. Med. Chem., 2007, 15, 4674-4699.
46. Molecular Operating Environment (MOE), 2013.08; Chemical Computing Group Inc., 1010 Sherbooke St. West, Suite #910, Montreal, QC, Canada, H3A 2R7, 2013.
47. J. Pandit, D. E. Danley, G. K. Schulte, S. Mazzalupo, T. A. Pauly, C. M. Hayward, E. S. Hamanaka, J. F. Thompson and H. J. Harwood, Jr., J. Biol. Chem., 2000, 275, 30610-30617.
56. A. Sharma, R. Kumar, N. Sharma, V. Kumar and A. K. Sinha, Adv. Synth. Catal., 2008, 350, 2910-2920.
57. A. Jahnke, B. Beile and H. Meier, Helv. Chim. Acta, 2011, 94, 2111-2124.
58. R. Bandari, T. Hoeche, A. Prager, K. Dirnberger and M. R. Buchmeiser, Chem. Eur. J., 2010, 16, 4650-4658, 54650/4651-S4650/4612.
59. K. Tajima, L.-S. Li and S. I. Stupp, J. Am. Chem. Soc., 2006, 128, 5488-5495.
63. K. Ahmad, N. F. Thomas, M. R. Mukhtar, I. Noorbatcha, J.-F. Faizal Weber, M. A. Nafiah, S. S. Velu, K. Takeya, H. Morita, C.-G. Lim, A. H. A. Hadi and K. Awang, Tetrahedron, 2009, 65, 1504-1516.
64. D. F. Taber and C. G. Nelson, J. Org. Chem., 2006, 71, 8973-8974.
65. T. Yamamoto and T. Yamakawa, J. Org. Chem., 2009, 74, 3603-3605.
66. Z. Tong, P. Gao, H. Deng, L. Zhang, P.-F. Xu and H. Zhai, Synlett, 2008, 3239-3241.
67. J.-J. Tang, G.-J. Fan, F. Dai, D.-J. Ding, Q. Wang, D.-L. Lu, R.-R. Li, X.-Z. Li, L.-M. Hu, X.-L. Jin and B. Zhou, Free Radical Biol. Med., 2011, 50, 1447-1457.
68. N. C. Ganguly and P. Mondal, Synthesis, 2010, 3705-3709.
69. T. Kohno, K. Togashi and N. Fukamiya, Nat. Prod. Res., 2007, 21, 606-615.
76. C. Lion, C. S. Matthews, M. F. G. Stevens and A. D. Westwell, J. Med. Chem., 2005, 48, 1292-1295.
82. Y.-J. Shang, Y.-P. Qian, X.-D. Liu, F. Dai, X.-L. Shang, W.-Q. Jia, Q. Liu, J.-G. Fang and B. Zhou, J. Org. Chem., 2009, 74, 5025-5031.
83. N. M. Cullinane, J. Chem. Soc., Trans., 1923, 123, 2053-2060.
84. C. A. Faler and M. M. Joullie, Org. Lett., 2007, 9, 1987-1990.
85. R. W. Taft, Jr., E. Price, I. R. Fox, I. C. Lewis, K. K. Andersen and G. T. Davis, J. Am. Chem. Soc., 1963, 85, 709-724.
86. N. Taniguchi, J. Org. Chem., 2006, 71, 7874-7876.
87. J.-H. Li, B.-X. Tang, L.-M. Tao, Y.-X. Xie, Y. Liang and M.-B. Zhang, J. Org. Chem., 2006, 71, 7488-7490.
88. S. E. Denmark and C. R. Butler, J. Am. Chem. Soc., 2008, 130, 3690-3704.
89. H. Lebel, M. Davi, S. Diez-Gonzalez and S. P. Nolan, J. Org. Chem., 2007, 72, 144-149.

1. A method of treating hyperlipaemia, diseases or conditions associated with hyperlipidaemia/hypercholesterolaemia, or trypansomal diseases, said method comprising administering to a subject in need of such treatment a therapeutically effective amount of a compound of Formula I 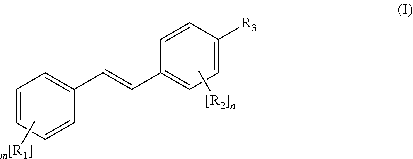
wherein:
m is 0, 1 or 2;
each R₁ group present is independently selected from halo, cyano, nitro, hydroxyl, (1-3C)alkyl, (1-3C)haloalkyl, (1-3C)alkoxy, (1-3C)haloalkoxy, —S—R^{a}, —S(O)—R^{a}, —S(O)₂—R^{a}, —C(O)NR^{a}R^{b}, —NR^{a}C(O)R^{b}, —C(O)R^{a}, —C(O)OR^{a}, —OC(O)R^{a}, —S(O)₂—NR^{a}R^{b} or —NR^{a}S(O)₂R^{b}, wherein R^{a} and R^{b} are each independently selected from hydrogen or (1-3C)alkyl;
n is 0, 1 or 2;
each R₂ group present is independently selected from halo, cyano, nitro, hydroxyl, (1-3C)alkyl, (1-3C)haloalkyl, (1-3C)alkoxy, (1-3C)haloalkoxy, —S—R^{c}, —S(O)—R^{c}, —S(O)₂—R^{c}, —C(O)NR^{c}R^{d}, —NR^{c}C(O)R^{d}, —C(O)R^{c}, —C(O)OR^{c}, —OC(O)R^{c}, —S(O)₂—NR^{c}R^{d} or —NR^{c}S(O)₂R^{d}, wherein R^{c} and R^{d} are each independently selected from hydrogen or (1-3C)alkyl; and
R₃ is a group of the formula:
—X¹-L-X²-Q
wherein:
X¹ is absent or selected from —O—, —N(R^{e})—, —N(R^{e})—C(O)—, —C(O)—N(R^{e})—, —N(R^{e})C(O)N(R^{f})—, —C(O)—, —C(O)O—, —OC(O)—, —S—, —SO—, —SO₂—, —S(O)₂N(R^{e})—, —N(R^{e})SO₂—, wherein R^{e} and R^{f} are each independently selected from hydrogen or (1-3C)alkyl;
L is —(CR^{g}R^{h})ₚ—, wherein p is 3, 4 or 5, and R^{g} and R^{h} are at each occurrence independently selected from hydrogen, (1-2C)alkyl or (1-2C)haloalkyl, or R^{g} and R^{h} can be linked such that, together with the carbon atom to which they are attached, they form a (3-6C)cycloalkyl ring;
X² is absent or selected from —O—, —N(Rⁱ)—, —N(Rⁱ)—C(O)—, —C(O)—N(Rⁱ)—, —N(R^{j})C(O)N(Rⁱ)—, —C(O)—, —C(O)O—, —OC(O)—, —S—, —SO—, —SO₂—, —S(O)₂N(Rⁱ)—, —N(Rⁱ)SO₂—, wherein Rⁱ and R^{j} are each independently selected from hydrogen or (1-3C)alkyl; and
Q is selected from hydrogen or (1-6C)alkyl, wherein, when Q is (2-6C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;
or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)haloalkyl, (1-2C)alkoxy or (1-2C)haloalkoxy;
with the proviso that Q and Rⁱ are not both ethyl when Xⁱ is —O—, p is 3, R^{g} and R^{h} are both hydrogen; and X² is —N(Rⁱ)—;
or a pharmaceutically acceptable salt or solvate thereof.

2. The method according to claim 1, wherein said method is for treating hypercholesterolaemia, atherosclerosis, coronary arterial diseases, cerebral ischemia, intermittent claudication or gangrene.

3. The method according to claim 1, wherein said method is for treating hypercholesterolaemia.

4. The method according to claim 1, wherein Q and Rⁱ are both hydrogen or methyl when X¹ is —O—, p is 3, R^{g} and R^{h} are both hydrogen; and X² is —N(Rⁱ)—.

5. The method according to claim 1, wherein Q is methyl and Rⁱ is hydrogen or methyl.

6. The method according to claim 1, wherein m is 0 or 1.

7. The method according to claim 6, wherein each R₁ group present is independently selected from halo, cyano, nitro, hydroxyl, (1-2C)alkyl, (1-2C)haloalkyl, (1-2C)alkoxy, (1-2C)haloalkoxy, —S—R^{a}, —S(O)—R^{a}, —S(O)₂—R^{a}, —C(O)NR^{a}R^{b}, —NR^{a}C(O)R^{b}, —C(O)R^{a}, —C(O)OR^{a}, —OC(O)R^{a}, —S(O)₂—NR^{a}R^{b} or —NR^{a}S(O)₂R^{b}, wherein R^{a} and R^{b} are each independently selected from hydrogen or (1-2C)alkyl.

8. The method A compound for use in treatment according to any one of the preceding claims claim 6, wherein each R₁ group present is independently selected from fluoro, chloro, cyano, nitro, hydroxyl, (1-2C)alkyl, (1-2C)haloalkyl, (1-2C)alkoxy, (1-2C)haloalkoxy.

9. The method according to claim 7, wherein n is 0 or 1.

10. The method according to claim 9, wherein R₂ group present is independently selected from halo, cyano, nitro, hydroxyl, (1-2C)alkyl, (1-2C)haloalkyl, (1-2C)alkoxy, (1-2C)haloalkoxy, —S—R^{c}, —S(O)—R^{c}, —S(O)₂—R^{c}, —C(O)NR^{c}R^{d}, —NR^{c}C(O)R^{d}, —C(O)R^{c}, —C(O)OR^{c}, —OC(O)R^{c}, —S(O)₂—NR^{c}R^{d} or —NR^{c}S(O)₂R^{d}, wherein R^{c} and R^{d} are each independently selected from hydrogen or (1-2C)alkyl.

11. The method according to claim 9, wherein each R₂ group present is independently selected from fluoro, chloro, cyano, nitro, hydroxyl, (1-2C)alkyl, (1-2C)haloalkyl, (1-2C) alkoxy, (1-2C)haloalkoxy.

12. The method according to claim 10, wherein R₃ is a group of the formula:
—X¹-L-X²-Q
wherein:
X¹ is selected from —O—, —N(R^{e})—, —N(R^{e})—C(O)—, —C(O)—N(R^{e})—, —C(O)O—, —OC(O)—, —S—, —SO—, —SO₂—, —S(O)₂N(R^{e})—, —N(R^{e})SO₂—, wherein R^{e} is independently selected from hydrogen or (1-2C)alkyl;
L is —(CR^{g}R^{h})ₚ—, wherein p is 3, 4 or 5, and R^{g} and R^{h} are at each occurrence independently selected from hydrogen, (1-2C)alkyl or (1-2C)haloalkyl, or R^{g} and R^{h} can be linked such that, together with the carbon atom to which they are attached, they form a (3-6C)cycloalkyl ring;
X² is selected from —O—, —N(Rⁱ)—, —N(Rⁱ)—C(O)—, —C(O)—N(Rⁱ)—, —C(O)O—, —OC(O)—, —S—, —SO—, —SO₂—, —S(O)₂N(Rⁱ)—, —N(Rⁱ)SO₂—, wherein Rⁱ is independently selected from hydrogen or (1-2C)alkyl; and
Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl;
or Q and Rⁱ are linked such that, together with the nitrogen atom to which they are attached, they form a 4, 5 or 6-membered heterocyclic ring which optionally comprises one or two further heteroatoms selected from the group consisting of O, S, or N, and wherein the heterocyclic ring is optionally further substituted with one or more substituents selected from oxo, halo, hydroxyl, cyano, amino, (1-2C)alkyl, (1-2C)halo alkyl, (1-2C)alkoxy or (1-2C)haloalkoxy.

13. The method according to claim 10, wherein R₃ is a group of the formula:
—X¹-L-X²-Q
wherein:
X^{l} is —O—;
L is —(CR^{g}R^{h})ₚ—, wherein p is 3, and R^{g} and R^{h} are at each occurrence independently selected from hydrogen or methyl;
X² is selected from —O— or —N(Rⁱ)—, wherein Rⁱ is independently selected from hydrogen or (1-2C)alkyl; and
Q is selected from hydrogen or (1-4C)alkyl, wherein, when Q is (2-4C)alkyl, it is optionally further substituted with one or more substituents selected from halo, hydroxyl, cyano, amino, (1-2C)haloalkyl, (1-2C)alkoxy or (1-2C)haloalkoxy and when Q is methyl it is optionally further substituted with one or more substituents selected from halo, cyano or (1-2C)haloalkyl.

14. The method according to claim 10, wherein R₃ is a group of the formula
—X¹-L-X²-Q
wherein:
X¹ is —O—;
L is —(CR^{g}R^{h})ₚ—, wherein p is 3, and R^{g} and R^{h} are at each occurrence independently selected from hydrogen or methyl;
X² is selected from —O— or —N(Rⁱ)—, wherein Rⁱ is independently selected from hydrogen or methyl; and
Q is selected from hydrogen or methyl.

15. The method according to claim 10, wherein R₃ is a group of the formula:
—X¹-L-X²-Q
wherein:
X¹ is —O—;
L is —(CR^{g}R^{h})ₚ—, wherein p is 3, and R^{g} and R^{h} are at each occurrence independently selected from hydrogen or methyl;
X² is —O—; and
Q is hydrogen or methyl.

16. The method according to claim 1, wherein
(E)-N,N-Dimethyl-3-(4-styrylphenoxy)propan-1-amine;
(E)-1-((4-Methylpentyl)oxy)-4-styrylbenzene;
(E)-1-(3-Methoxypropoxy)-4-styrylbenzene;
(E)-3-(4-(4-Methoxystyryl)phenoxy)-N,N-dimethylpropan-1-amine;
(E)-3-(4-(3-Methoxystyryl)phenoxy)-N,N-dimethylpropan-1-amine;
(E)-3-(4-(2-Methoxystyryl)phenoxy)-N,N-dimethylpropan-1-amine;
(E)-N,N-Dimethyl-3-(4-(2-(naphthalen-2-yl)vinyl)phenoxy) propan-1-amine;
(E)-3-(4-(4-Fluorostyryl)phenoxy)-N,N-dimethylpropan-1-amine;
(E)-3-(4-(3-Fluorostyryl)phenoxy)-N,N-dimethylpropan-1-amine;
(E)-N,N-Dimethyl-3-(4-(4-nitrostyryl)phenoxy)propan-1-amine;
(E)-N,N-Dimethyl-3-(4-(3-nitrostyryl)phenoxy)propan-1-amine;
(E)-N,N-Dimethyl-3-(4-(2-nitrostyryl)phenoxy)propan-1-amine;
(E)-3-(2-Fluoro-4-styrylphenoxy)-N,N-dimethylpropan-1-amine;
(E)-N-Methyl-3-(4-styrylphenoxy)propan-1-amine;
(E)-4-Styrylphenol;
(E)-3-(4-Styrylphenoxy)propan-1-ol;
(E)-1-Methoxy-4-styrylbenzene;
or a pharmaceutically acceptable salt or solvate thereof.

17. A compound having the structural formula I 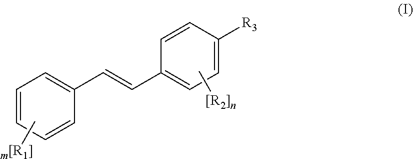
wherein m, R₁, n, R₂ and R₃ are each as defined in claim 1, with the proviso that the compound is not one of the following:
(E)-N,N-Dimethyl-3-(4-styrylphenoxy)propan-1-amine;
(E)-3-(4-Styrylphenoxy)propan-1-ol; and
(E)-N,N-Diethyl-2-(4-styrylphenoxy)propan-1-amine.

18. A compound according to claim 17, with the proviso that:
(i) Q and Rⁱ are not both ethyl when X¹ is —O—, p is 3, R^{g} and R^{h} are both hydrogen; and X² is —N(12¹)—; and
(ii) Q is not hydrogen when X¹ and X² is —O—.

19. A compound according to claim 18, wherein m and n are not both 0.

20. A compound according to claim 17, wherein the compound is selected from any one of the following:
(E)-1-((4-Methylpentyl)oxy)-4-styrylbenzene;
(E)-1-(3-Methoxypropoxy)-4-styrylbenzene;
(E)-3-(4-(4-Methoxystyryl)phenoxy)-N,N-dimethylpropan-1-amine;
(E)-3-(4-(3-Methoxystyryl)phenoxy)-N,N-dimethylpropan-1-amine;
(E)-3-(4-(2-Methoxystyryl)phenoxy)-N,N-dimethylpropan-1-amine;
(E)-N,N-Dimethyl-3-(4-(2-(naphthalen-2-yl)vinyl)phenoxy) propan-1-amine;
(E)-3-(4-(4-Fluorostyryl)phenoxy)-N,N-dimethylpropan-1-amine;
(E)-3-(4-(3-Fluorostyryl)phenoxy)-N,N-dimethylpropan-1-amine;
(E)-N,N-Dimethyl-3-(4-(4-nitrostyryl)phenoxy)propan-1-amine;
(E)-N,N-Dimethyl-3-(4-(3-nitrostyryl)phenoxy)propan-1-amine;
(E)-N,N-Dimethyl-3-(4-(2-nitrostyryl)phenoxy)propan-1-amine;
(E)-3-(2-Fluoro-4-styrylphenoxy)-N,N-dimethylpropan-1-amine;
(E)-N-Methyl-3-(4-styrylphenoxy)propan-1-amine;
or a pharmaceutically acceptable salt or solvate thereof.

21. (canceled)

22. A pharmaceutical composition comprising a compound according to claim 17, or a pharmaceutically acceptable salt thereof, and one or more pharmaceutically acceptable excipients.

23. The method according to claim 1, wherein said method is for treating sleeping sickness or chagas disease.

24. A method of treating hyperlipaemia, diseases or conditions associated with hyperlipidaemia/hypercholesterolaemia, or trypansomal diseases, said method comprising administering to a subject in need of such treatment a therapeutically effective amount of a pharmaceutical composition of claim 22.
